(19) United States
(12) Patent Application Publication
(10) Pub. No.: US 2024/0033208 A1
(43) Pub. Date: Feb. 1, 2024

(54) BOTANICAL EXTRACT BLEND FOR USE IN SKIN CARE

(71) Applicant: Shaklee Corporation, Pleasanton, CA (US)

(72) Inventors: Teodoro T. Ianiro, Concord, CA (US); Jillian Friedman, Kingston, NY (US)

(73) Assignee: Shaklee Corporation, Pleasanton, CA (US)

(21) Appl. No.: 18/227,138

(22) Filed: Jul. 27, 2023

Related U.S. Application Data
(60) Provisional application No. 63/392,663, filed on Jul. 27, 2022.

(51) Int. Cl. A61K 8/9789; A61Q 19/08; A61K 8/06

(52) U.S. Cl. CPC A61K 8/9789; A61Q 19/08; A61K 8/06; A61K 2800/80

(57) ABSTRACT

Topical compositions containing a decolorized muscadine grape pomace extract, a water extract of Chardonnay grape seed, and resveratrol are disclosed, as well as methods of use thereof. Also disclosed are methods of preparing the topical compositions.

### CROSS REFERENCE TO RELATED APPLICATIONS

This application claims priority to U.S. Provisional Application No. 63/392,663, filed Jul. 27, 2022, which is incorporated by reference in its entirety.

### FIELD

This disclosure relates to topical compositions and their use for improving the appearance of skin.

### BACKGROUND

Fruits and vegetables possess considerable antioxidant capacity and can influence multiple cellular signaling cascades. Polyphenols are considered important for mediating the health benefits of the Mediterranean diet and red wine. Grapes are a particularly rich source of polyphenols.

Muscadine grapes contain several bioactive polyphenolic compounds, including flavonoids (such as flavonols, anthocyanins, and flavanones, as well as flavan-3-ols and oligomers thereof known as proanthocyanidins) and non-flavonoids (such as phenolic acids, tannins and stilbene derivatives, for example resveratrol). The protective and anti-inflammatory effects of flavonoids are believed to be due to free radical scavenging, beneficial effects on cellular signaling pathways and gene expression, and selective interference with the cell division cycle of rapidly and abnormally proliferating mammalian cells. In addition, Chardonnay seed contains a relatively high content of epicatechin, a flavanol monomer that is reported to mediate beneficial actions on human endothelial function and may underlie, at least in part, the effects of cocoa and grape seed extract on improving circulatory function. Resveratrol is a polyphenol and a powerful antioxidant prominently found in grape skin. There are two main isomers of resveratrol: cis- and trans-resveratrol.

A need remains for topical compositions that include grape extracts and resveratrol, that act on the skin, and can be included in topical compositions, such as, but not limited to, lotions and serums.

### SUMMARY

Topical compositions including a decolorized muscadine grape pomace extract, a water extract of Chardonnay grape seed, and resveratrol are disclosed herein. In some examples, the resveratrol is 90% to 99% trans-resveratrol, for example, at least 90%, at least 95%, at least 98%, etc., trans-resveratrol. The disclosed topical compositions are of use for improving skin appearance. The disclosed topical compositions are also of use for inhibiting elastase or matrix metalloproteinase-1 (MMP-1), decreasing advanced glycation end product (AGE) formation or decreasing AGE content of collagen, or decreasing collagen breakdown in skin.

In one non-limiting example, the disclosed topical composition includes: (a) a decolorized muscadine grape pomace extract at about 0.025% to about 0.4% by weight of the topical composition (w/w), a water extract of Chardonnay grape seed at about 0.006% to about 0.1% by weight of the topical composition (w/w), and resveratrol at about 0.018% to about 0.3% by weight of the topical composition (w/w); or (b) the decolorized muscadine grape pomace extract at about 0.0025% to about 0.04% by weight of the topical composition (w/w), the water extract of Chardonnay grape seed at about 0.006% to about 0.1% by weight of the topical composition (w/w), and resveratrol at about 0.018% to about 0.3% by weight of the topical composition (w/w). For both (a) and (b), the decolorized muscadine grape pomace extract includes, by percentage dry weight of the extract, about 9% to about 10% polyphenols, less than 4% monosaccharides, and condensed tannins are less than 10% of total polyphenols of the decolorized muscadine grape pomace extract. In addition, for both (a) and (b), the water extract of Chardonnay grape seed includes, by percentage dry weight of the water extract, 38-50% polyphenols, 9-12% fiber, 1-2% protein, less than 1% lipids, and 25-30% sugars.

Also disclosed is a topical composition that includes a blend of decolorized muscadine grape pomace extract, water extract of Chardonnay grape seed, and resveratrol, in a ratio of 1-10:1-5:2.5-7.5, respectively. The decolorized muscadine grape pomace extract includes, by percentage dry weight of the extract, about 9% to about 10% polyphenols, less than about 4% monosaccharides, and condensed tannins are less than about 10% of the total polyphenols of the decolorized muscadine grape pomace extract. The water extract of Chardonnay grape seed includes, by percentage dry weight of the water extract, 38-50% polyphenols, 9-12% fiber, 1-2% protein, <1% lipids, and 25-30% sugars. In some examples, the resveratrol is 90% to 99% trans-resveratrol, for example, at least 90%, at least 95%, at least 98%, etc., trans-resveratrol.

The topical composition can be a cosmetic, for example, a skin care product. In some examples, the topical composition is a serum or a lotion. The topical composition can further include additional ingredients, such as one or more vitamins, additional botanical extracts, fats or oils, surfactants, emollients, emulsifiers, humectants, prebiotics, probiotics, and enzymes.

In some aspects, when applied to the skin of a subject, the topical composition inhibits elastase or matrix metalloproteinase-1 (MMP-1), decreases advanced glycation end product (AGE) formation or AGE content of collagen, decreases collagen breakdown, and/or improves the appearance of skin, relative to a suitable control (e.g., a measurement obtained from the subject prior to application of the topical composition).

Also disclosed are methods of preparing the disclosed topical compositions.

The foregoing and other features of the disclosure will become more apparent from the following detailed description, which proceeds with reference to the accompanying figures.

### BRIEF DESCRIPTION OF THE FIGURES

FIG. 1 is a schematic illustration of a process for making the decolorized muscadine grape pomace extract, which is disclosed in PCT Publication No. WO 2018/017608.

FIG. 2 is a schematic depiction of a system for carrying out the process of FIG. 1. See also PCT Publication No. WO 2018/017608.

FIGS. 3A-3B are comparative traces of chromatographic profiles of polyphenols in a precursor muscadine pomace extract before (FIG. 3A) and the decolorized muscadine grape pomace extract (FIG. 3B). The desired polyphenolic profile of the precursor extract is retained, while the level of condensed tannins is greatly lowered in the decolorized extract. See also PCT Publication No. WO 2018/017608.

FIG. 4 is a schematic illustration of the process for making the water extract of Chardonnay grape seed, which is disclosed in PCT Publication No. WO 2015/184291.

FIG. 5 is a schematic illustration of an exemplary process for extracting resveratrol from Polygonum cuspidatum root.

FIG. 6 is a bar graph of the percent inhibition of elastase using topical compositions.

FIG. 7 is a bar graph of the percent inhibition of MMP-1 (collagenase) using topical compositions.

FIG. 8 is a bar graph of AGE prevention by topical compositions.

FIG. 9 is a bar graph of AGE reduction by topical compositions.

FIGS. 10A-10B a bar graph of results from a 2,2-diphenyl-1-picrylhydrazyl (DPPH) assay that measures free radical scavenging (shown in Trolox equivalents), using topical compositions.

FIG. 11 is a bar graph of the results from a cyclobutene pyrimidine dimer (CPD) assay, showing dimer formation in genomic DNA using MatTek EPIDERM® tissue.

### DETAILED DESCRIPTION

Topical compositions are disclosed that contain blends of grape extracts and resveratrol with enhanced skin care benefits. A combination of resveratrol with polyphenols enhances the expression of antioxidant genes, particularly with muscadine grapes. The topical compositions disclosed herein combine polyphenols from all aspects of a grape source (e.g., skin, pomace, pulp, juice, and seed) to achieve synergistic antioxidant effects. The disclosed topical compositions exhibit multiple activities, such as, but not limited to: antioxidant activity, AGE inhibition, elastase and collagenase inhibition, and cyclobutane pyrimidine dimer prevention, indicating that these topical compositions are of benefit in skin care.

### I. Terms

Unless otherwise noted, technical terms are used according to conventional usage. The term “or” refers to a single element of stated alternative elements, or a combination of two or more elements, unless the context clearly indicates otherwise. As used herein, the singular forms “a,” “an,” and “the,” refer to both the singular as well as plural, unless the context clearly indicates otherwise. For example, the term “an extract” includes singular or plural extracts, and can be considered equivalent to the phrase “at least one extract.” Further, as used herein, “comprises” means “includes.” Thus, “comprising A or B,” means “including A, B, or both A and B,” without excluding additional elements. All percentages and ratios are calculated by weight unless otherwise indicated. The term “about” refers to within 5% of an indicated value, for example, “about” 100 grams refers to 95 grams to 105 grams, or “about” 0.5% refers to 0.475% to 0.525%, unless clearly indicated otherwise.

Although methods and materials similar or equivalent to those described herein can be used in the practice or testing of the present disclosure, suitable methods and materials are described herein. To facilitate review of the various aspects of this disclosure, the following explanations of terms are provided:

Administration (or application): To provide or give a subject an agent by any effective route. Exemplary routes of administration include, but are not limited to: topical, oral, injection (such as subcutaneous, intramuscular, intradermal, intraperitoneal, intravenous, and intratumoral), sublingual, transdermal, intranasal, topical, and inhalation routes. Administration includes application of a topical composition to the skin.

Advanced glycation end product (AGE): Compounds formed by nonenzymatic glycation and oxidation of proteins and lipids. Connective tissue aging is characterized by a progressive accumulation of AGEs. In the context of skin aging, AGEs promote fibroblast apoptosis, inhibit the synthesis of collagen, and accelerate the degradation of collagen. An increase in the AGE content in the skin is associated with skin yellowing or browning, poor elasticity, and deeper wrinkles.

Antioxidant: A substance that inhibits oxidation or removes oxidizing agents, such as free radicals.

Chardonnay Grape: A green-skinned grape variety of Vitis vinifera used in the production of white wine. A water extract of chardonnay seeds is disclosed in U.S. Pat. No. 10,709,751, which is herein incorporated by reference in its entirety.

Collagen: The main structural protein in the extracellular matrix found in the body's various connective tissues. As the main component of connective tissue, it is the most abundant protein in mammals, making up from 25% to 35% of the whole-body protein content. Collagen consists of amino acids bound together to form a triple helix of elongated fibril known as a collagen helix. The collagen protein is composed of a triple helix, which generally consists of two identical chains (α1) and an additional chain that differs slightly in its chemical composition (α2). There are two classes of collagen proteins, fibrillar and non-fibrillar. Fibrillar collagen includes Type I, II, III, V, and XI collagen, and non-fibrillar collagen includes Type IV, VI, VII, IX, X XII, XIV, XV, XVII, XVIII, XIX, and XXI. Collagen has been widely used in cosmetic surgery, as a healing aid for burn patients for reconstruction of bone and a wide variety of dental, orthopedic, and surgical purposes.

Control: A reference standard. A control may be any suitable positive or negative control. Several non-limiting examples are provided for illustrative purposes. In some examples, a suitable control is a base line measurement (e.g., a measurement obtained prior to applying a topical composition), or an empty vehicle control. In another example, a suitable control is a historical control or standard reference value or range of values (such as a previously obtained control sample, or group of samples that represent baseline or “normal” values). A difference between a test sample and a control can be an increase or conversely a decrease. The difference can be a qualitative (e.g., subjective observations) or quantitative (e.g., objective measurements). In some examples, the difference is statistically significant (e.g., a P-value greater than 0.05, for example, greater than 0.01 or 0.001, as determined by a statistical test, such as a Student's T-Test or ANOVA).

Cosmetic: A composition that changes the appearance of a subject. A cosmetic composition can be applied to the body, such as the skin or hair, to change the appearance. Cosmetics include, but are not limited to, serum, lotions, powders, lipsticks, and other preparations for beautifying the skin.

Cosmetically Acceptable Vehicle: Suitable cosmetically acceptable vehicles (carriers) (e.g., pharmaceutically acceptable carriers) have been described, for example, in Remington: The Science and Practice of Pharmacy, 23rd Ed. (Academic Press), 2021 (ISBN 9780128200070), which describes compositions and formulations suitable for delivery of topical compositions. The use of pharmaceutically acceptable carriers does not imply that that product so made is useful only for pharmaceutical purposes. Rather it implies that the product is suitable for administration (e.g., topical application) to a subject. In particular examples, the vehicle is a carrier for a topical composition, such as a liquid, gel, foam, cream, ointment, or lotion. Particularly useful vehicles are those that are pharmaceutically or cosmetically acceptable for topical applications, such as one or more aqueous systems, glycerin, C₁ alcohols, fatty alcohols, fatty ethers, fatty esters, polyols, glycols, vegetable oils, mineral oils, liposomes, laminar lipid materials, silicone oils, water, or any combinations thereof. The carrier can also be a variety of existing skin serums, lotions, gels, creams, ointments, toners, cleansers, moisturizers, or sunscreens.

Decolorized: A “decolorized” extract refers to one having less color than a reference, such as a precursor extract of grapes, and it does not require an absence of coloration. A “decolorized” extract as used in this specification refers to the extract having a lowered level of condensed tannins and a lesser dark coloration of the type associated with condensed tannins in red grapes, red grape extracts, and red wine. “Decolorized muscadine pomace grape extracts” are disclosed in PCT Publication No. WO 2010/014870, PCT Publication No. WO 2010/014873, U.S. Pat. Nos. 8,568,804, 9,132,162, 9,173,916, and 10,967,034, all of which are herein incorporated by reference in their entirety.

Decreasing (or Reducing): A negative change relative to a control value, such as a decrease of at least 10%, at least 15%, at least 20%, at least 25%, at least 30%, at least 40%, at least 50%, at least 60%, at least 70%, at least 80%, at least 90%, at least 95%, at least 98%, at least 99%, or at least 100% decrease as compared to a control value. A decrease can be within a range, for example, about 25% to 100%, about 25% to 98%, about 25% to 95%, about 25% to 90%, about 25% to 80%, about 25% to 70%, about 25% to 50%, about 50% to 100%, about 75% to 100%, about 90% to 100%, about 95% to 100%, about 98% to 100%, about 99% to 100%, about 50% to 75%, about 50% to 80%, about 50% to 90%, about 50% to 95%, about 50% to 98%, about 75% to 80%, about 75% to 90%, about 75% to 95%, or about 75% to 98%.

Effective Amount: An amount of a composition that achieves a desired response, such as, but not limited to, antioxidant activity, AGE inhibition, elastase and collagenase inhibition, cyclobutane pyrimidine dimer inhibition, or improved appearance of skin. The effective amount can be administered in a single dose, or in several doses, for example daily. However, the effective amount can depend on the subject, the condition of the subject, and the manner of administration.

Elastase: A member of the chymotrypsin family of proteases. Elastase is primarily responsible for the breakdown of elastin, but can also cleave collagen, fibronectin and other extracellular matrix proteins. Elastin is vital for giving elasticity to arteries, lungs, ligaments and skin. In terms of anti-ageing, inhibitors of elastase can be useful to prevent loss of skin elasticity and thus skin sagging.

Emollient: A cosmetic preparation used for protecting, moisturizing, and lubricating the skin. Moisturizers modify the rate of water loss, with active ingredients of moisturizers falling into one of two categories: occlusives and humectants. Moisturizers often contain water, which acts as a temporary hydration agent as well as a way for the absorption of some components and evaporation of the moisturizer. Occlusives form a coating on the surface of the skin, keeping moisture from escaping. The more occlusive the formulation, the greater the effect. Ointments are more occlusive than aqueous creams, which are more occlusive than lotion. Humectants are substances that absorb water. Humectants can absorb water from the air and moisturize skin when the humidity is greater than 70%, but more commonly can draw water from the dermis into the epidermis.

Enzyme: A protein that act as biological catalysts (biocatalysts). Enzymes differ from most other catalysts by being much more specific, as they use only a defined substrate and generate a certain product. An enzyme's activity decreases markedly outside its optimal temperature and pH, and many enzymes are denatured when exposed to excessive heat, losing their structure and catalytic properties. Enzymes can be from fruit acids. In some examples, the enzyme is from pineapple, papaya, banana, and/or a citrus fruit.

Excipient: An inactive substance used as a carrier for the active ingredients of a composition. Excipients can include substances that are used to bulk up formulations with active ingredients, allow for convenient and accurate dosage, stabilize the active ingredients, and make the delivery system optically and/or organoleptically acceptable. Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like.

Extract: A solution or other preparation of at least some of the active ingredients of a plant or one or more of its parts, such as its fruit or seeds. An extract initially obtained by solvent extraction may be converted into a dried form and still be considered an “extract.” An “aqueous” or “water” extract refers to an extract obtained by solvent extraction with water, with the exclusion of other solvents (such as ethanol).

Fats and oils: Esters of glycerol with three fatty acids (also called triglycerides or triacylglycerols), or a mixture of such compounds. Although the terms “oils” and “fats” are often used interchangeably, they are usually used to distinguish triglycerides in the liquid state at ambient temperatures (oils) from those in the solid state (fats). They are commonly of vegetable origin (e.g., palm oil, rapeseed oil, soyabean oil, olive oil, cocoa butter, etc.) or animal origin (e.g. pork lard, beef tallow, fish oils, etc.) as well as from animal milk fats. The fatty acids found in common oils and fats are composed of long carbon and hydrogen chains, typically containing from 8 to 20 carbon atoms, mainly with even numbers of carbon atoms, although animal fats also contain significant levels of odd-chain fatty acids. They have a methyl (CH₃) group at one end and a carboxylic acid (COOH) at the other. It is this carboxylic acid group that reacts with the hydroxyl groups on the glycerol molecule to form the ester linkages of the triacylglycerol molecule.

Flavonoids: A class of plant secondary metabolites. Flavonoids constitute an important group of dietary polyphenolic compounds that are widely distributed in plants. More than 4000 chemically unique flavonoids have been identified in plant sources, such as fruits, vegetables, legumes, nuts, seeds, herbs, spices, flowers, as well as in beverages such as tea, cocoa, beer, wine, and grape juice.

On a dry weight basis, grape seeds contain about 4% to about 8% flavonoids. Flavonoids in grape seeds refer primarily to flavan-3-ols, specifically (+)-catechin, (−)-epicatechin, and (−)-epicatechin 3-gallate, and complexes thereof. The flavan-3-ols in grape seeds are present in monomeric, oligomeric or polymeric forms. Two or more chemically linked flavan-3-ol monomers are called proanthocyanidins or oligomeric proanthocyanidins (“OPCs”), which includes procyanidins and prodelphinidins. OPCs containing two monomers are called dimers, three monomers are called trimers, four monomers are called tetramers, five monomers are called pentamers, etc. The oligomers have chain lengths of 2 to 10; polymers represent components with chain lengths greater than 10. Thus, oligomers in grape seed extracts include, for instance, dimers and trimers, and there is evidence that the polymers can have as many as 50 to 100 units.

Flavonoids are present in all parts of the grape, including the skin, juice and pulp, and not just in the grape seed. In order for polyphenolic compounds to be used commercially as a grape extract, including as grape seed or grape pomace extracts, these compounds have to be separated from grapes in a more concentrated form. Extensive research suggests that grape extracts, such as grape seed extracts, are beneficial in many areas of health because of its promoting youthful skin, cell health, elasticity, and flexibility. Other studies have shown that proanthocyanidins help to protect the body from sun damage, to improve vision, to improve flexibility in joints, arteries, and body tissues such as the heart, and to improve blood circulation by strengthening capillaries, arteries, and veins.

Increasing (or Improving): A positive change relative to a control value, such as an increase of at least 25%, at least 50%, at least 75%, at least 100%, at least 200%, at least 300%, at least 400%, or at least 500% as compared to the control value. An increase can be within a range, for example, about 25% to 500%, about 25% to 400%, about 25% to 300%, about 25% to 200%, about 25% to 100%, about 25% to 75%, about 25% to 50%, about 50% to 500%, about 75% to 500%, about 100% to 500%, about 200% to 500%, about 300% to 500%, about 400% to 500%, about 50% to 100%, about 50% to 200%, about 50% to 300%, about 50% to 400%, about 50% to 500%, about 100% to 200%, about 100% to 300%, about 100% to 400%, about 100% to 500%, or about 250% to 500%.

Improving the appearance of skin: Effecting a visually and/or physically measurable benefit in skin appearance and/or feel. Improving the appearance of skin can include increasing skin firmness, increasing skin elasticity, increasing skin hydration, and/or decreasing appearance of wrinkles.

Lotion: A topical preparation, including a moisturizing agent, intended for application to the skin. By contrast, creams and gels have higher viscosity, typically due to lower water content. Lotions are usually suspensions of solids, and typically include a liquid oily emulsion of the oil-in-water type. In some examples, the lotion is about 73% water based upon the weight of the lotion composition (w/w).

Matrix Metalloproteinase-1 (MMP-1): An extracellular enzyme capable of degrading substrates, such as collagen, gelatin, and entactin. In human skin, MMP-1 is the major protease capable of initiating fragmentation of collagens, predominantly type I and III collagens. MMP-1 is a member of a larger family of enzymes that cleave extracellular matrix proteins called matrix-degrading metalloproteinases (MMPs). MMPs are constitutively elevated in aged dermis, and are major mediators of skin connective tissue damage that is observed in aged human skin.

Moisturizer: A skin care product that is formulated to add moisture to the external layers of the skin, and can act as a vehicle for topical delivery of ingredients to the skin. A moisturizer can have different modes of action, including by forming a film on the surface of the skin to reduce moisture loss, by attracting water vapor from the air to add moisture, or by adding moisturizing factors to the skin surface. A moisturizer can be an emulsion, or a viscous semi-solid.

Muscadine Grape (Vitus rotundifolia): A grape native to the southeastern United States. The phytochemical constituents of the whole muscadine grape differ from Vitis vinifera. Muscadines have a higher total phenolic content distinguished by high ellagic, gallic, and flavonoid glycoside concentrations. The presence of ellagic acid in muscadine grapes is unique and is found in the form of free ellagic acid, ellagic acid glycosides, methoxylated derivatives and ellagitannins. Another unique feature is the anthocyanin chemistries observed in muscadines. Present as 3,5-diglucosides (as opposed to 3-glucosides) of delphinidin, cyanidin, petunidin, peonidin, and malvidin in non-acylated forms, these compounds and the natural color influence from other anthocyanins present within the grape impart a dark purple color to juice and pomace obtained from the purple varieties.

Polyphenols: A large family of naturally occurring organic compounds characterized by multiples of phenol units. Polyphenols from grapes and cocoa have been found to enhance both cardiovascular function and cognitive health. Flavanols (also called flavan-3-ols) represent the majority of grape seed and cocoa polyphenols; this class of phenolic compounds ranges from monomeric species such as catechin and epicatechin to oligomers (often termed proanthocyanidins) to polymers (often termed tannins or condensed tannins). The term “phenolic” is used interchangeably with the term polyphenol in the art and in this specification. Polyphenols are present in multiple parts of the grape, including in the skin, juice and pulp. The highest concentration of polyphenols in a grape resides in the grape seeds.

Pomace: The skins, seeds, and pulp remaining following juice extraction. In one example a pomace extract is a bronze muscadine pomace extract, a purple muscadine pomace extract or a combination thereof. Many different varieties of muscadine grape pomaces are available as starting materials, and they include female (pistillate) varieties such as Black Beauty, Black Fry, Darlene, Fry, Higgins, Jumbo, Scuppernong, Sugargate, Summit, Supreme, and Sweet Jenny, and self-fertile varieties such as Carlos, Cowart, Dixieland, Dixie Red, Fry Seedless, Magnolia, Nesbitt, Noble, Redgate, Regale and Sterling. Muscadine pomace contains phenolic compounds, including gallic acid and ellagic acid, having antioxidant properties.

Prebiotic: Nondigestible fiber compounds that pass undigested through the upper part of the gastrointestinal tract and stimulate the growth or activity of advantageous bacteria, such as in the colon or another tissue. Common prebiotics used in food manufacturing include beta-glucan from oats and inulin from chicory root.

Probiotic: Live microorganisms that when administered in adequate amounts confer a health benefit on the host. A probiotic is safe to consume or use in cosmetic applications. Live probiotic cultures are part of fermented dairy products, other fermented foods, and probiotic-fortified foods. Additionally, lactic acid bacteria (LABs), which are food fermenting bacteria, have the ability to prevent food spoilage and can improve the value of the products they inhabit.

Purified: The term purified does not require absolute purity; rather, it is intended as a relative term. Thus, for example, a purified substance is one in which the substance is more enriched than the substance in its natural environment. In one example, a preparation is purified such that the substance (for example, trans-resveratrol), represents at least 5% (such as, but not limited to, at least 10%, 20%, 30%, 40%, 50%, 60%, 70%, 80%, 90%, 95%, 98% or 99%) of the total content of the preparation.

Range: With respect to ranges, the term “in the range of x to y” or “from x to y” includes any value between x and y, as well as the endpoints x and y.

Resveratrol: A polyphenolic compound found in peanuts, grapes, red wine, and some berries. Resveratrol is a potent antioxidant. It occurs in both trans and cis molecular configurations. Both cis- and trans-resveratrol can also occur as glucosides, (i.e., bound to a glucose molecule). Resveratrol is described on public databases, such as PubChem (PubChem ID: 445154). The preferred IUPAC name of resveratrol is 5-[(E)-2-(4-Hydroxyphenyl)ethen-1-yl]benzene-1,3-diol. It is also referred to as 3,5,4′-trihydroxy-trans-stilbene. Resveratrol is commercially available from various manufacturers, for example, “Giant Knotweed Rhizome P.E. 98%” from World-Way Biotech, Inc. (China).

Serum: A skin care product that is formulated to deliver ingredients deeply into the skin, that does not include a moisturizer. Serums generally contains higher concentrations of active ingredients, and are applied directly to clean skin prior to application of a moisturizer. A serum can have a low viscosity and a higher proportion of water than oil. A serum composition can include synthetic or natural oils, including poly-alkyl siloxanes, as well as an emulsifying agent, alcohols such as ethanol, a lipid, a pH balancing agent, and other pharmaceutically acceptable carriers. Ingredients that can improve absorption of the serum composition into deeper layers of the skin can also be added, such as pentylene glycol ethanol, dipentaerythrityl hexacaprylate or hexacaprate, and propanediol. In one example, the serum composition is about 85% water based upon the weight of the serum composition (w/w).

Skin care composition: A topical composition having ingredients that can improve the health, aesthetic and/or cosmetic appearance of skin.

Skin firmness, elasticity, and hydration: Specific measurements of skin appearance.

Firmness refers to tautness of the skin. Moisturizers can increase skin firmness by enhancing skin water content. Elasticity refers to the skin's ability to stretch and bounce back into place. As skin loses elasticity, it starts to sag and wrinkle. Two proteins, collagen and elastin, are primarily responsible for providing skin with the required amount of firmness and elasticity. Hydration refers to the water content in the skin, which leads cells to swell and be plump. Well hydrated skin is supple and soft.

Subject: Living multi-cellular vertebrate organisms, a category that includes both human and veterinary subjects, such as a companion animal, including a cat, dog, or horse. In some examples, the subject is a human.

Surfactant: A compound that lowers the surface tension (or interfacial tension) between two liquids, between a gas and a liquid, or between a liquid and a solid. Surfactants may act as detergents, wetting agents, emulsifiers, foaming agents, or dispersants. Surfactants are usually organic compounds that are amphiphilic. Surfactants will diffuse in water and adsorb at interfaces between air and water or at the interface between oil and water, in the case where water is mixed with oil.

Tannins: Naturally occurring polyphenolic biomolecules that are found in plants, such as red grape seeds, seed and stems. In terms of their chemical composition, plant tannins are divided into the hydrolyzable tannins and the condensed tannins or flavonoids. The hydrolyzable tannins are polymerized simple phenolic substances, such as esters of gallic acid and its dimers (digallic acid, ellagic acid). The hydrolyzable tannin can be further divided into gallotannins that yield gallic acid after hydrolysis, and ellagitannins that release ellagic acid after hydrolysis. In contrast to the hydrolyzable tannins, the condensed tannins are not decomposable by hydrolysis. On the contrary, when subjected to heating in an acidic medium they progressively polymerize and form amorphous anthocyanin pigments of red color, or insoluble yellow-brown products, of high molecular mass, called phlobaphenes.

Unit dose: A physically discrete unit containing a predetermined quantity of an active material calculated to individually or collectively produce a desired effect. A single unit dose or a plurality of unit doses can be used to provide the desired effect or activity.

Vitamin: An organic molecule that is an essential micronutrient that an organism needs in small quantities for the proper functioning of its metabolism. Essential nutrients cannot be synthesized in the organism, either at all or not in sufficient quantities, and therefore must be obtained through the diet. Vitamins include Vitamins A, B₁, B₂, B₃, B₅, B₆, B₇, B₉, B₁₂, C, D, E and K. These Vitamins are classified as either water-soluble or fat-soluble. In humans there are four fat-soluble (A, D, E, and K) and nine water-soluble (the eight B vitamins and vitamin C) vitamins.

In case of conflict, the present specification, including explanations of terms, will control. In addition, the materials, methods, and examples are illustrative only and not intended to be limiting.

### II. Overview

Disclosed are topical compositions that include a decolorized muscadine grape pomace extract, a water extract of Chardonnay grape seed, and resveratrol. In some examples, the topical composition includes:
(a) a decolorized muscadine grape pomace extract at about 0.025% to about 0.4% by weight of the topical composition (w/w), a water extract of Chardonnay grape seed at about 0.006% to about 0.1% by weight of the topical composition (w/w), and resveratrol at about 0.018% to about 0.3% by weight of the topical composition (w/w); or
(b) a decolorized muscadine grape pomace extract at about 0.0025% to about 0.04% by weight of the topical composition (w/w), a water extract of Chardonnay grape seed at about 0.006% to about 0.1% by weight of the topical composition (w/w), and resveratrol at about 0.018% to about 0.3% by weight of the topical composition (w/w).

For both (a) and (b) above, the decolorized muscadine grape pomace extract includes, by percentage dry weight of the extract, about 9% to about 10% polyphenols, of which less than 10% of the total polyphenols are condensed tannins, and less than 4% monosaccharides. The decolorized muscadine grape pomace extract and methods of producing the decolorized muscadine grape pomace extract is described herein and, for example, in U.S. Pat. No. 10,967,034 B2, herein incorporated by reference in its entirety. In addition, for both (a) and (b) above, the water extract of Chardonnay grape seed includes, by percentage dry weight of the water extract, 38-50% polyphenols, 9-12% fiber, 1-2% protein, <1% lipids, and 25-30% sugars. The water extract of Chardonnay grape seed and methods of producing the water extract of Chardonnay grape seed is described herein and, for example, in U.S. Pat. No. 10,709,751, herein incorporated by reference in its entirety.

In some examples, the topical composition includes the decolorized muscadine grape extract at about 0.10% w/w, the water extract of Chardonnay grape seed at about 0.025% w/w, and resveratrol at about 0.075% w/w. In some examples, the topical composition includes the decolorized muscadine grape extract at about 0.05% w/w, the water extract of Chardonnay grape seed at about 0.0125% w/w, and resveratrol at about 0.0375% w/w. In some examples, the topical composition includes the decolorized muscadine grape pomace extract at about 0.01% w/w, the water extract of Chardonnay grape seed at about 0.025% w/w, and resveratrol at about 0.075% w/w. In further examples, the topical composition includes the decolorized muscadine grape pomace extract at about 0.005% w/w, the water extract of Chardonnay grape seed at about 0.0125% w/w, and resveratrol at about 0.0375% w/w.

In some aspects, the topical composition includes the decolorized muscadine grape pomace extract, the water extract of Chardonnay grape seed, and resveratrol, in a ratio of 1-10:1-5:2.5-7.5, respectively. In some examples, the topical composition incudes the decolorized muscadine grape pomace extract, the water extract of Chardonnay grape seed, and resveratrol, in a ratio of 10:1-5:2.5-7.5, respectively. In a non-limiting example, the topical composition includes the decolorized muscadine grape pomace extract, the water extract of Chardonnay grape seed, and resveratrol, in a ratio of 10:2.5:7.5, respectively. In some examples, the topical composition includes the decolorized muscadine grape pomace extract, the water extract of Chardonnay grape seed, and resveratrol, in a ratio of 1:1-5:2.5-7.5, respectively. In a non-limiting example, the topical composition includes the decolorized muscadine grape pomace extract, the water extract of Chardonnay grape seed, and resveratrol, in a ratio of 1:2.5:7.5, respectively.

The topical composition can be a cosmetic, for example, a skin care product. In some examples, the topical composition is a serum or a lotion. The topical composition can further comprise additional ingredients, such as one or more vitamins, additional botanical extracts, fats or oils, surfactants, emollients, emulsifiers, humectants, probiotics, and enzymes.

In some aspects, the topical composition inhibits elastase or matrix metalloproteinase-1 (MMP-1), decreases advanced glycation end product (AGE) formation or AGE content of collagen, decreases collagen breakdown, or improves the appearance of skin, relative to a suitable control.

Also disclosed are methods of inhibiting elastase or matrix metalloproteinase-1 (MMP-1), decreasing advanced glycation end product (AGE) formation or AGE content of collagen, or decreasing collagen breakdown in skin of a subject, by applying to the skin of the subject an effective amount of a disclosed topical composition. Methods of improving the appearance of skin by applying an effective amount of a disclosed topical composition, for a period of time sufficient to improve the appearance of skin, are also disclosed. In some examples, improving the appearance of skin includes increasing skin firmness, increasing skin elasticity, or increasing skin hydration. The topical composition is applied for a period of time sufficient to achieve a desired effect, such as inhibiting elastase or matrix metalloproteinase-1 (MMP-1), decreasing advanced glycation end product (AGE) formation or AGE content of collagen, decreasing collagen breakdown, or improving appearance of the skin. In some examples, the topical composition is a serum or a lotion.

Also disclosed are methods of preparing a topical composition, which includes mixing a topical composition base with the decolorized muscadine grape pomace extract, the water extract of Chardonnay grape seed, and resveratrol to achieve a desired concentration (such as a concentration of the decolorized muscadine grape pomace extract, the water extract of Chardonnay grape seed, and resveratrol, respectively, as disclosed herein).

### III. Description of Several Examples

A. Topical Compositions

Disclosed herein are topical compositions that include a decolorized muscadine grape pomace extract, a water extract of Chardonnay grape seed, and resveratrol. In some examples, the resveratrol is at least 50% trans-resveratrol, for example, at least 60%, at least 65%, at least 70%, at least 75%, at least 80%, at least 85%, at least 90%, at least 95%, at least 98%, or at least 99% trans-resveratrol. In a non-limiting example, the resveratrol is at least 90% trans-resveratrol. In a further non-limiting example, the resveratrol is at least 95% trans-resveratrol, for example, at least 98% trans-resveratrol. The topical compositions are useful for improving the appearance of skin, for example, by increasing skin firmness, increasing skin elasticity, or increasing skin hydration. In some aspects, the topical compositions inhibit elastase or matrix metalloproteinase-1 (MMP-1), decrease advanced glycation end product (AGE) formation or AGE content in collagen, or decrease collagen breakdown in skin.

i. The Decolorized Muscadine Grape Pomace Extract

The decolorized muscadine grape pomace extract, and method of making the decolorized muscadine grape pomace extract, have been described, for example, in U.S. Pat. No. 10,967,034 B2, which is herein incorporated by reference in its entirety.

The decolorized muscadine pomace extract has a lower level of condensed tannin content as compared to a muscadine pomace extract that has not been decolorized. Tannins are widely distributed in many types of plants. Tannins can be found in leaf, bud, seed, root, and stem tissues. Tannins are found in monocots (44 families) and dicots (180 families) and are found in gymnosperms and angiosperms. Decolorized plant extracts have a lower condensed tannin content as compared to non-decolorized precursor extracts of any of these plants that contain condensed tannins. In certain examples, the decolorized muscadine grape pomace extract is made from a liquid muscadine pomace extract that is a combined extract of bronze and purple muscadine grapes, having an enhanced solubility of ellagic acid (which is unique to muscadine grapes). The combined muscadine grape pomace precursor extract obtained by any of the processes disclosed herein, has levels of condensed tannins that impart dark coloration to the extract.

As discussed in applicant's disclosures of PCT Publication No. WO 2010/014870, PCT Publication No. WO 2010/014873, U.S. Pat. Nos. 8,568,804, 9,132,162 and 9,173,916 (all five of which are also incorporated herein by reference), muscadine extracts with improved ellagic acid solubility can be obtained by combining bronze and purple muscadine pomace extracts. Various methods of making the combined extracts were disclosed, such as combining a bronze muscadine pomace extract with a purple muscadine pomace extract to produce a muscadine pomace extract, wherein the ratio of bronze muscadine pomace extract to purple muscadine pomace extract ranges from about 0.1 to about 10 (weight to weight), such as about 0.3 to about 3 (weight to weight). In certain examples, the decolorized muscadine grape pomace extract is prepared from such a combined precursor extract. The combined precursor extract can be made by separate extraction of bronze and purple muscadine grapes with subsequent combination of the extracts, or by simultaneous extraction of bronze and purple muscadine grapes combined in desired ratios.

In some aspects, the ratio of bronze to purple muscadine pomace extract in the precursor liquid extract ranges from about 0.1 to about 10, such as about 0.3 to about 3. For example, the ratio of bronze muscadine pomace extract to purple muscadine pomace extract is about 2.75 to about 1 (weight to weight), about 2.5 to about 1 (weight to weight), about 2.25 to about 1 (weight to weight), about 2 to about 1 (weight to weight), about 1.5 to about 1 (weight to weight), or about 1 to about 1 (weight to weight). In other examples, the ratio is about 10 to about 1, about 7.5 to about 1, or about 5 to about 1. As used in this paragraph, the term “about” is defined as ±0.5. In a particular example, the ratio of bronze muscadine pomace extract to purple muscadine pomace extract is about 2.25 to about 1 (weight to weight). In a particular example, the ratio of bronze muscadine pomace extract to purple muscadine pomace extract is 2.25 to 1 (weight to weight).

In certain aspects, the combined precursor muscadine (Vitis rotundifolia) pomace liquid extract has a polyphenol content of at least 2%. For example, the polyphenol content is at least 3%, at least 3.5%, at least 4%, at least 4.5%, at least 5%, at least 6%, at least 7%, at least 8%, at least 10%, at least 12%, or at least 14%. In addition, the final simple sugar (monosaccharide) content of the decolorized extract is quite low (less than about 4 to less than about 5%) and thus suitable for use in a topically applied product. In some examples, the decolorized muscadine pomace extract includes less than about 5% monosaccharides by weight, for example, less than about 4%, less than about 3%, less than about 2%, or less than about 1% monosaccharides by weight. The liquid pomace precursor extracts can be obtained by any extraction method, such as pressing under pressure or extracting with a solvent. In a particular example, solvent extraction is used, for example with alcohol, water (such as heated water), or a combination of alcohol and water.

The extract can further be fermented to remove extracted sugars. In one example, fermentation is performed following extracting the bronze muscadine pomace and purple muscadine pomace but prior to combining the bronze muscadine pomace extract with the purple muscadine pomace extract to produce a liquid muscadine pomace extract. In other examples, fermentation is performed after combining the bronze muscadine pomace extract with purple muscadine pomace extract in the desired post extraction ratio (such as, but not limited to, at about a 2:25 to about 1 bronze to purple ratio).

Fermentation may be performed by any suitable, known method. For example, yeast and yeast nutrients can be added to the pomace and fermentation continued until the residual sugar content is converted to ethanol. In one example, two pounds of yeast are added per 1000 gallons of 1X (unconcentrated) extract; fermentation is typically complete after three days. In other examples, the amount and/or strain of yeast and duration and temperature of fermentation may vary according to individual methods. In some examples, enzymes are used to clarify and/or settle residues or to improve extraction yield in the pomace extracts. Examples of such enzymes include pectinase or a blend of enzymes from Aspergillus niger that are commercially available from sources such as Scott Laboratories. These enzymes may be added to the pomace extract before or during fermentation.

In some examples, the bronze muscadine pomace extracts and purple muscadine pomace extracts are filtered prior to and/or following fermentation. Filtration can be performed according to known methods. In a particular example, extracts are filtered through sieves of appropriate mesh size, such as USP mesh (typically 120 mesh) or a similar cloth filter (for example filters commercially available from Millipore Corporation).

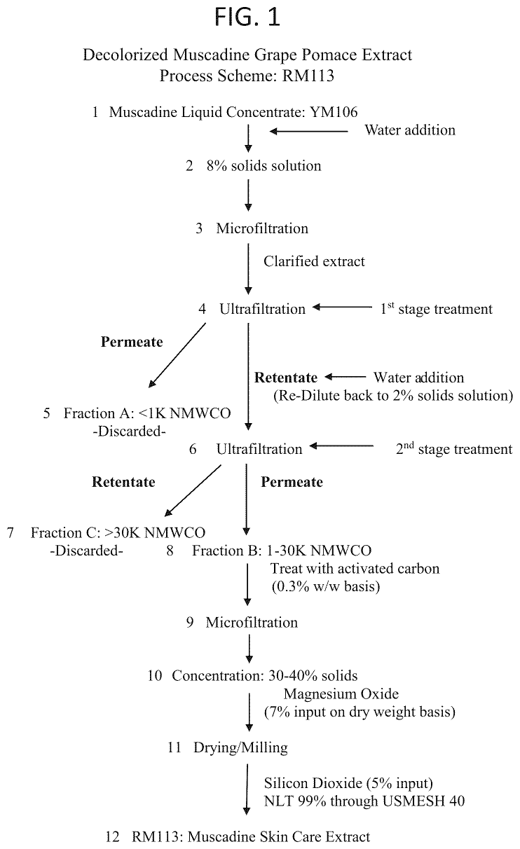

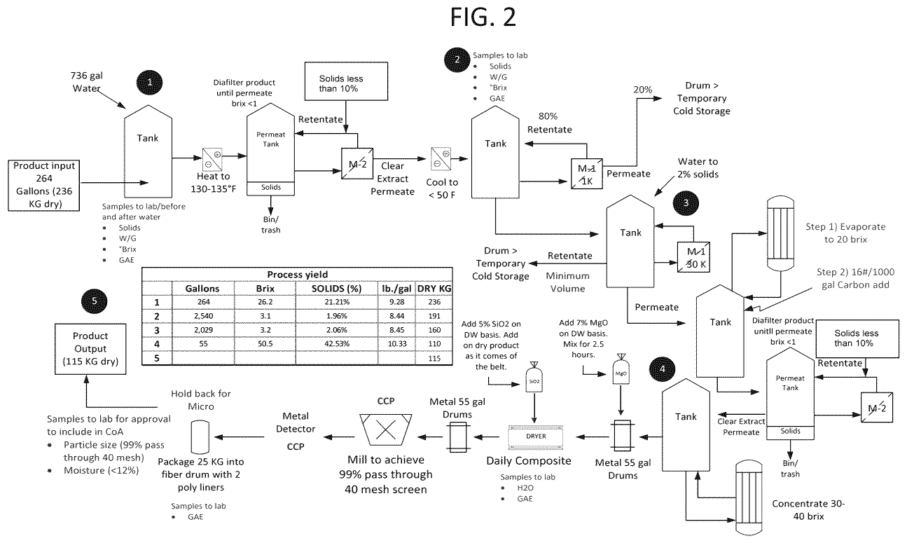

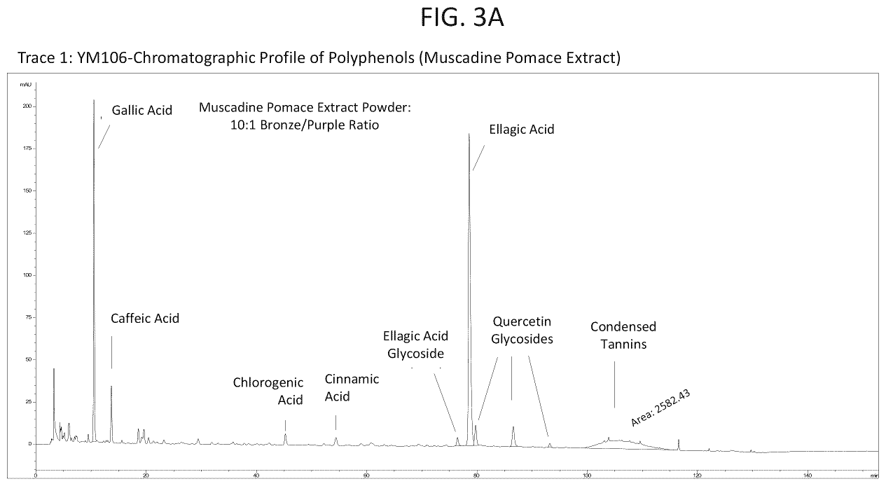

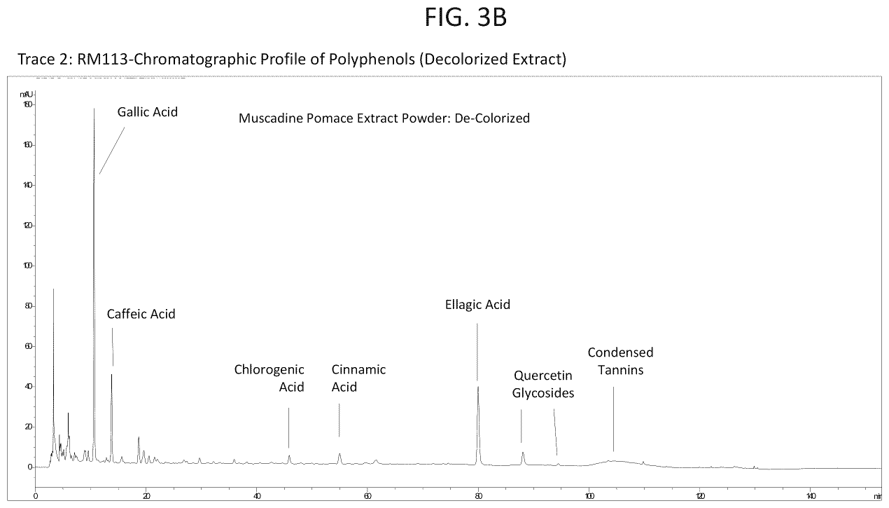

FIG. 1 provides a process scheme for making the decolorized muscadine grape pomace extract. The decolorized muscadine grape pomace extract is sometimes referred to as “RM113.” In some examples, the decolorized muscadine grape pomace extract is produced by the process described in FIG. 1. In some examples, the decolorized muscadine grape pomace extract is RM113. The process for producing the extracts is disclosed in PCT Publication No. WO 2018/209117, incorporated herein by reference.

An example of a process for making the decolorized muscadine grape pomace extract is shown herein in FIG. 1. A precursor material is a muscadine pomace extract liquid concentrate 1 (YM106). In this example, the concentrate 1 is about 40 to about 45% solids, as described in U.S. Pat. Nos. 8,512,771 and 9,173,916. Concentrate 1 is diluted to less than about 10% solids by adding purified water to produce an about 8% solids solution 2 (the percent solids can range from about 2 to about 10%). Solution 2 is heated to about 130 to about 135 T and passed through a microfiltration device 3 to clarify the material. The microfiltration process is more efficient when the precursor material is diluted to a very low level of solids (about 2 to about 5%) and heating also helps facilitate the process. In this specific example, the microfiltration device 3 is a mechanical filter of 0.45-micron pore size and NMWCO of 200,000 daltons (or 200 kDa) which removes microorganisms and suspended particles from solution in preparation for a subsequent ultrafiltration treatment 4. The clarified extract is quickly cooled down to <50° F. in a holding tank before starting the 1^{st} stage ultrafiltration step.

The cooled and clarified extract is then subjected to ultrafiltration treatment 4 with standard nominal molecular weight cutoff ultrafiltration membrane of 1000 daltons (or 1 kDa) yielding a permeate (Fraction A) at 5. Fraction A includes compounds having molecular weights of less than about 1000 and contains mainly organic acids, sugars, and minerals but surprisingly contains less than about 1% phenolic compounds. These phenolic compounds include trace amounts of phenolic acids and flavonoids such as gallic acid and quercetin. Although molecules having a molecular weight of up to about 1,000 daltons are able to permeate the ultrafiltration membrane, it was unexpectedly found that this fraction contained a very low phenolic acid content, perhaps due to physicochemical interactions between the phenolic acids and other molecules, possibly fiber, contained in the precursor material. This unexpected finding can be obtained with the use of standard nominal molecular weight cutoff ultrafiltration membranes as low as about 500 daltons and up to about 5000 (or 5 kDa). Fraction A could serve as a “muscadine” type flavor component in other products, but this material is discarded in the process of preparing the decolorized muscadine grape pomace extract used for the topical compositions described herein.

The retentate (material that does not pass through the ultrafiltration membrane) remaining from the low molecular weight ultrafiltration (described above) is reconstituted in purified water to approximately 2% solids concentration and subjected to a second ultrafiltration process 6 with a 30 kDa cutoff ultrafiltration membrane. The resulting retentate 7 (Fraction C) is discarded and the permeate 8 (Fraction B) containing the decolorized muscadine pomace extract is retained for further processing into the disclosed topical compositions. Fraction C primarily contains polymerized condensed tannins, protein, and fiber. In contrast, Fraction B is enriched with polyphenols (about 9 to about 10% dry weight), low in sugars (less than about 4% dry weight) and greatly reduced in polymeric condensed tannin content (undergoing a minimum reduction of about 70% of the initial condensed tannin content). Although molecules having a molecular weight of up to about 30,000 daltons are able to permeate the ultrafiltration membrane, it was unexpectedly found that at least 70% of the polymeric condensed tannins remained in Fraction C, possibly owing to physicochemical interactions. This unexpected finding may result from the use of standard nominal molecular weight cutoff ultrafiltration membranes as low as about 25,000 daltons (25 kDa) and up to about 100,000 daltons (or 100 kDa).

Fraction B also contains fiber (about 1.0%), protein (about 7.8%), fat (about 0.1%) and organic acids (about 16.0%). Fraction C, which is enriched in polymeric condensed tannins, could serve as a source of antioxidants for use in a variety of products, but this material is discarded in the preparation process of the decolorized muscadine grape pomace extract. At this point in the decolorization process, the muscadine fruit extract, Fraction B, has minimal sugar content (less than about 4% dry weight) and is greatly reduced in polymeric condensed tannins. Fraction B is useful for skin care products because it contains powerful polyphenols yet has little or no potential for brown discoloration owing to the removal of condensed tannins and its minimal sugar content. When introduced into various formulations for topical use, brown color undertones are not detected.

Permeate 8 (Fraction B) is further processed by treating it with activated carbon at an about 0.3% weight to weight basis to remove odiferous components that are undesirable in a skin care product (buttery/yeasty notes). After treatment with activated carbon, Fraction B is subjected to a final microfiltration process 9 to remove all traces of activated carbon. After microfiltration the solution is concentrated 10 to about 30 to about 40% solids before mineralization with magnesium oxide to facilitate a free-flowing powder after drying. Magnesium oxide is added in at an input of about 7.0% on a dry weight basis to the concentrate to completely facilitate the mineralization process, so as to form the magnesium salts of all the organic acids that exist in the extract. Finally, the product is subjected to drying/milling 11 to provide a uniform particle size and produce RM113. Silicon dioxide is added during this milling stage to prevent product caking.

Compared to the precursor material, Fraction B has a mass reduction of about 75 to about 80% yet only about 35 to about 40% polyphenol content reduction when subjected to the above-described process. Therefore, when comparing Fraction B to the precursor material, on an equivalent mass basis, the resulting reduction of non-tannic polyphenol content in Fraction B is only about 18 to about 20%. Thus, the predominant loss of polyphenols in Fraction B is attributed to the chemical class of polymeric condensed tannins; virtually no loss of other polyphenol chemical classes was detected. Thus, Fraction B contains, on average, about 9% muscadine polyphenols (predominantly non-tannic) whereas, the precursor material contains, on average, about 14% polyphenols consisting of both tannic and non-tannic muscadine polyphenols.

In some examples, the decolorized muscadine grape pomace extract includes, by percentage dry weight, about 9% to about 10% polyphenols and less than 4% monosaccharides. Condensed tannins are less than 10% of total polyphenols of the decolorized muscadine grape pomace extract. Additional information regarding the nutritional profile of the decolorized muscadine grape pomace extract is provided in Table 1. The nutritional profile can be compared to the precursor extract (a “reference” extract), see, PCT Publication No. WO 2018/209117.

TABLE 1
Nutritional Profile of the Decolorized Muscadine Grape Pomace Extract

| | Decolorized Extract |
|---|---|
| Compound Class | (% dry weight) |
| Protein | 7.8 |
| Fat | 0.1 |
| Ash (Inorganic Constituents) | 18.9 |
| Sugar Acids | 16.5 |
| Soluble Fiber | 0.9 |
| Insoluble Fiber | 0.1 |
| Organic Acids | 16.0 |
| Sugar Alcohols | 8.8 |
| Polyphenols | 9 (Avg. G.A.E.) |
| Simple Sugars | 3.5 |
| Moisture | 10.0 |
| Amino Acids | 7.8 |
| “other” | 0.6 |
| Total | 100.0 |

In some examples, the decolorized muscadine grape pomace extract has the nutritional profile described in Table 1.

Table 2 shows, on a dry weight basis, the total polyphenol content of the decolorized muscadine grape pomace extract, and further divides the polyphenol content into condensed tannin content and content of polyphenols other than condensed tannins.

TABLE 2
Polyphenol Content of the Decolorized Muscadine Grape Pomace Extract
Phenolic Profiling

| Phenolic | | % of total | Analysis |
|---|---|---|---|
| Class | mg/g extract | polyphenols | Method |
| All other | 84.0 | 93.3 | (G.A.E. minus |
| Polyphenols | | | condensed |
| | | | tannins)* |
| Condensed | 6.0 | 6.7 | n-Butanol- |
| Tannins | | | HCL assay |
| Total | 90 | 100 | G.A.E Assay |
| Polyphenols | | | |

*Gallic Acid Equivalents assay (Total Polyphenols via UV/VIS) − Condensed Tannins assay via UV/VIS = non-condensed tannins content.

As shown in Tables 3 and 4, the decolorized muscadine grape pomace extract retains the core polyphenols found in muscadine grape. The phenolic profiling and polyphenol profile shown in these tables can be compared to the precursor extract (a “reference” extract), see PCT Publication No. WO 2018/209117.

TABLE 3
Polyphenol Profiles of the Decolorized Muscadine Grape Pomace Extract

| | | % of | |
|---|---|---|---|
| | mg/g | identified | |
| Phenolic Class | extract | polyphenols | Analysis Method |
| Ellagic Acid | 0.56 | 2.6 | HPLC/MS Quantitation w/ |
| Ellagic Acid | 0.67 | 3.1 | Respective Standards |
| Glycosides | | | |
| Gallic Acid | 6.55 | 30.7 | |
| Quercetin/ | 0.55 | 2.6 | |
| Glycosides | | | |
| Gallotannins | 2.16 | 10.1 | Hydrochloric Acid Hydrolysis |
| Ellagitannins | 1.64 | 7.7 | HPLC/MS Quantitation: Ellagic |
| | | | Acid and Methyl Gallate increase |
| Proantho- | 6.33 | 29.7 | C-18 S.P.E. |
| cyanidins | | | Vanillin-Sulfuric Acid Assay & |
| (Oligomeric/ | | | n-Butanol-HCL assay |
| Polymeric) | | | |
| Anthocyanins | 0.99 | 4.6 | HPLC/MS Quantitation (520 nm) |
| | | | Cyanidin-3,5-Diglucoside Std |
| Catechins | 0.60 | 2.8 | HPLC/MS Quantitation (280 nm) |
| | | | (+/−)-Catechin Std |
| Phenolic Acids | 1.29 | 6.1 | HPLC/MS Quantitation (254 nm) |
| | | | Chlorogenic Acid |
| Experimental | 21.34 | 100 | |
| Total | | | |

TABLE 4
Core Muscadine Grape Polyphenols

Phenolic Acids
Ellagic Acid (aglycone & glycosides)
Gallic Acid
Chlorogenic, Caffeic, Cinnamic, and p-Coumaric
Flavonoids
Anthocyanosides: diglycoside linkages
Anthocyanidins: delphnidin, petunidin, malvidin
Flavonols: quercitin, myricetin, and kaempferol
Flavanols (flavan-3-ol's): catechin, epicatechin
Stilbenes
trans-Resveratrol, piceatannol(tetrahydroxy)
Tannins
Hydrolyzable: ellagitannins, gallotannins
Condensed: proanthocyanidins (oligomeric/polymeric)

In some aspects, the topical composition includes the decolorized muscadine grape pomace extract at about 0.00025% to about 4.0% by weight of the topical composition (w/w), for example, about 0.00025% to about 3.5% w/w, about 0.00025% to about 3% w/w, about 0.00025% to about 2.5% w/w, about 0.00025% to about 2.0% w/w, about 0.00025% to about 1.5% w/w, about 0.00025% to about 1.0% w/w, about 0.00025% to about 0.5% w/w, about 0.00025% to about 0.25% w/w, about 0.00025% to about 0.125% w/w, about 0.00025% to about 0.0625% w/w, about 0.00025% to about 0.025% w/w, about 0.00025% to about 0.01% w/w, about 0.00025% to about 0.005% w/w, about 0.0005% to about 4.0% w/w, about 0.001% to about 4.0% w/w, about 0.0025% to about 4.0% w/w, about 0.005% to about 4.0% w/w, about 0.01% to about 4.0% w/w, about 0.025% to about 4.0% w/w, about 0.05% to about 4.0% w/w, about 0.1% to about 4.0% w/w, about 0.25% to about 4.0% w/w, about 0.5% to about 4.0% w/w, about 1.0% to about 4.0% w/w, about 1.5% to about 4.0% w/w, about 2.0% to about 4.0% w/w, about 2.5% to about 4.0% w/w, about 3.0% to about 4.0% w/w, about 3.5% to about 4.0% w/w, about 0.001% to about 3% w/w, about 0.01% to about 3% w/w, about 0.025% to about 3% w/w, about 0.05% to about 3% w/w, about 0.1% to about 3% w/w, about 0.25% to about 3% w/w, about 0.5% to about 3% w/w, about 1.0% to about 3% w/w, about 2.0% to about 3% w/w, about 0.001% to about 2% w/w, about 0.01% to about 2% w/w, about 0.025% to about 2% w/w, about 0.05% to about 2% w/w, about 0.1% to about 2% w/w, about 0.25% to about 2% w/w, about 0.5% to about 2% w/w, about 1.0% to about 2% w/w, about 0.001% to about 1% w/w, about 0.01% to about 1% w/w, about 0.025% to about 1% w/w, about 0.05% to about 1% w/w, about 0.1% to about 1% w/w, about 0.25% to about 1% w/w, about 0.5% to about 1% w/w, about 0.001% to about 0.5% w/w, about 0.01% to about 0.5% w/w, about 0.025% to about 0.5% w/w, about 0.05% to about 0.5% w/w, about 0.1% to about 0.5% w/w, about 0.25% to about 0.5% w/w, about 0.001% to about 0.4% w/w, about 0.01% to about 0.4% w/w, about 0.025% to about 0.4% w/w, about 0.05% to about 0.4% w/w, about 0.1% to about 0.4% w/w, about 0.25% to about 0.4% w/w, about 0.00025% to about 0.04% w/w, about 0.0005% to about 0.04% w/w, about 0.001% to about 0.04% w/w, about 0.01% to about 0.04% w/w, about 0.025% to about 0.04% w/w, about 0.00025% to about 0.25% w/w, about 0.0005% to about 0.25% w/w, about 0.0025% to about 0.25% w/w, about 0.005% to about 0.25% w/w, about 0.05% to about 0.25% w/w, about 0.00025% to about 0.1% w/w, about 0.0005% to about 0.1% w/w, about 0.0025% to about 0.1% w/w, about 0.005% to about 0.1% w/w, or about 0.05% to about 0.1% w/w of the topical composition.

In some examples, the topical composition includes the decolorized muscadine grape pomace extract at about 0.025% to about 0.4% by weight of the topical composition (w/w). In some examples, the topical composition includes the decolorized muscadine grape pomace extract at about 0.05% to about 0.4% by weight of the topical composition (w/w). In some examples, the topical composition includes the decolorized muscadine grape pomace extract at about 0.05% to about 0.3% by weight of the topical composition (w/w). In some examples, the topical composition includes the decolorized muscadine grape pomace extract at about 0.05% to about 0.2% w/w.

In some examples, the topical composition includes the decolorized muscadine grape pomace extract at about 0.0025% to about 0.04% by weight of the topical composition (w/w). In some examples, the topical composition includes the decolorized muscadine grape pomace extract at about 0.005% to about 0.04% by weight of the topical composition (w/w). In some examples, the topical composition includes the decolorized muscadine grape pomace extract at about 0.005% to about 0.03% by weight of the topical composition (w/w). In some examples, the topical composition includes the decolorized muscadine grape pomace extract at about 0.005% to about 0.02% w/w.

In some examples, the topical composition includes the decolorized muscadine grape pomace extract at about 0.00025%, about 0.0005%, about 0.00075%, about 0.001%, about 0.0025%, about 0.005%, about 0.0075%, about 0.01%, about 0.025%, about 0.05%, about 0.075%, about 0.1%, about 0.25%, about 0.5%, about 0.75%, about 1.0%, about 1.25%, about 1.5%, about 01.75%, about 2.0%, about 2.25%, about 2.5%, about 2.75%, about 3.0%, about 3.25%, about 3.5%, about 3.75%, or about 4.0% by weight of the topical composition (w/w). In some examples, the topical composition includes the decolorized muscadine grape extract at about 0.10% w/w. In some examples, the topical composition includes the decolorized muscadine grape extract at about 0.05% w/w. In some examples, the topical composition includes the decolorized muscadine grape pomace extract at about 0.01% w/w. In a further example, the topical composition includes the decolorized muscadine grape pomace extract at about 0.005% w/w.

ii. The Water Extract of Chardonnay Grape Seed

The water extract of Chardonnay grape seed, and method of making the water extract of Chardonnay grape seed, have been described, for example, in U.S. Pat. No. 10,709,751, which is herein incorporated by reference in its entirety. The water extract of Chardonnay grape seed is sometimes referred to as “YG093” or “Fraction B.”

The disclosed extraction processes in U.S. Pat. No. 10,709,751 uses only a liquid water solvent (such as a pure water solvent) without the need for mineral acids to carry out the extraction. Extractions can also be performed below the boiling point of water (212° F.), and at atmospheric pressure. The beneficial components of the grape seeds are separated by sequential ultrafiltration steps into three fractions that are especially suited for different purposes: (A) a fraction containing mainly simple sugars and aromatic phenols for sweetening/flavoring purposes; (B) a fraction containing mainly low molecular weight flavanols, namely the monomeric and dimeric species that are useful for enhancing circulation; and (C) a fraction enriched in fiber and flavanol oligomers and polymers for imparting prebiotic effects. It is also notable that the latter two fractions (B and C) contain less than 1% lipid content as opposed to currently available commercial grape seed extracts that contain 2-3% lipid content. Typically, higher lipid content imparts a waxy, sticky characteristic that resists compression and impedes formulation of grape seed extract into tablets. Grape seed extracts with 2-3% lipid content tend to cause tablets to develop streaking and capping and are more incompatible with manufacturing machinery due to stickiness. Thus, grape seed extract formulations are most commonly found in capsules or in more expensive forms such as soft gels. The extremely low lipid content of the polyphenol enriched fractions described below allows formulation into tablets.

Chardonnay varietal is one of the most popular wines worldwide and thus abundant quantities of grape seed are generated. Moreover, Chardonnay seeds are reported to contain a relatively high content of epicatechin (Fuleki and Ricardo da Silva, J Agric Food Chem 45:1156-1160, 1997; Yilmaz and Toledo, J Agric Food Chem 52:255-260, 2004), a flavanol monomer that is reported to mediate beneficial actions on human endothelial function and may underlie, at least in part, the effects of cocoa and grape seed extract on circulatory function (Jimenez et al., J Agric Food Chem 60:8823-8830, 2012).

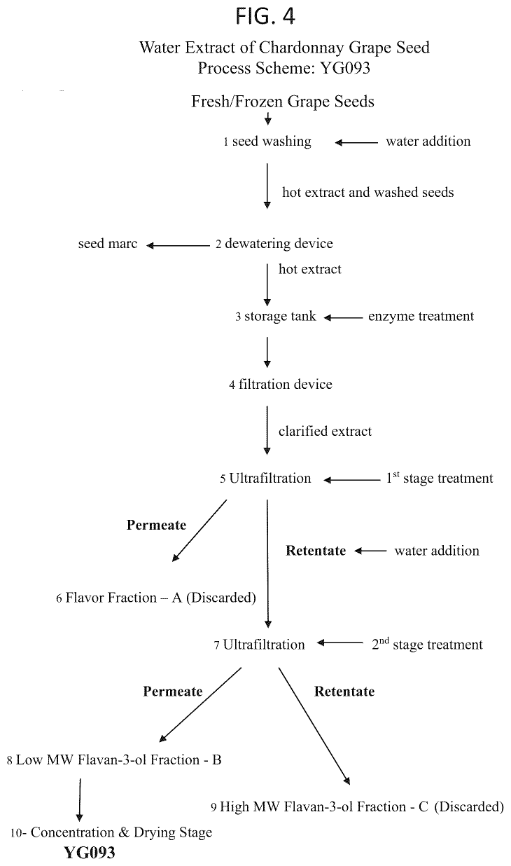

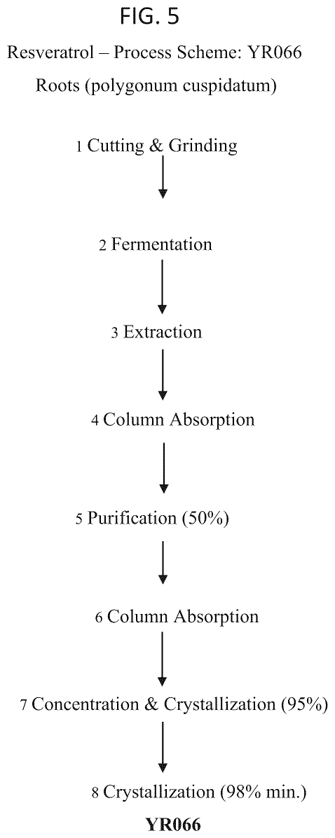

In a particular example of preparing the water extract of Chardonnay grape seed, washed Chardonnay grape seeds are extracted with hot water at a temperature of about 100-200° F., for example 120-195° F., such as 130-190° F. The initial extract is enzymatically treated and fine-filtered to substantially remove insoluble plant material and produce a clarified extract. The clarified extract is processed by a first ultrafiltration to obtain a first permeate and a first retentate, wherein larger molecular weight polymers are removed from the first permeate, and the first permeate comprises at least 80% sugars. The first retentate is reconstituted then processed by a second ultrafiltration to obtain a second permeate that contains polyphenols having an mdp of less than 3, for example less than 2.5, and a second retentate enriched in fiber and polyphenols, wherein the polyphenols have an mdp of greater than 5. The first ultrafiltration is performed with an ultrafiltration membrane having a molecular weight cutoff in the range of 1-5 kD, and the second ultrafiltration is performed with a membrane having a molecular weight cutoff in the range of 100-500 kD. An exemplary process of making the water extract of Chardonnay grape seed is shown herein in FIG. 4. In some examples, the water extract of Chardonnay grape seed is YG093 as shown in FIG. 4.

The Chardonnay seed extract is an extract obtained by extracting the Chardonnay seeds only with water, for example by water solvent extraction without the use of alcohol solvents (such as methanol or ethanol solvents), mineral acid solvents (such as sulfuric acid), or resins. Ultrafiltration produces an extract having a lipid content of less than 1%. In some examples, the water extract having a lipid content of less than 1% further contains flavanols having a mean degree of polymerization of less than 3, and in some examples less than 2.5.

The water extract of Chardonnay grape seed includes, by percentage dry weight, 38-50% polyphenols, 9-12% fiber, 1-2% protein, less than 1% lipids, and 25-30% sugars. The nutritional profile of the water extract of Chardonnay grape seed is provided in Table 5.

TABLE 5
Nutritional Profile of the Water Extract of Chardonnay Grape Seed

| Compound Class | % Dry Weight |
|---|---|
| Polyphenols (GAE) | 38-50% |
| Fiber | 9-12% |
| Protein | 1-2% |
| Lipid | <1% |
| Sugars | 25-30% |

Table 6 shows the distribution of low molecular weight (monomers, dimers, etc.) versus higher molecular weight (>10 units) flavanol species in the water extract of Chardonnay grape seed as measured by thiolysis and gel permeation chromatography (Gu et al., J Agric Food Chem 50:4852-4860, 2002; Hammerstone et al., J Agric Food Chem, 47:490-496, 1999).

TABLE 6
Flavanol Composition of the Water
Extract of Chardonnay Grape Seed

| Flavanol unit | % of Total Flavanols |
|---|---|
| Monomer (n = 1) | 76.13% |
| Dimer (n = 2) | 6.55% |
| Trimer (n = 3) | 3.03% |
| Tetramer (n = 4) | 2.37% |
| Pentamer (n = 5) | 1.89% |
| Hexamer (n = 6) | 0.91% |
| Heptamer (n = 7) | 0.45% |
| Octamer (n = 8) | 0.12% |
| Nonamer (n = 9) | Trace |
| Decamer (n = 10) | Trace |
| n > 10 | 8.55% |

In some aspects, the topical composition includes the water extract of Chardonnay grape seed at about 0.0006% to about 1.0% by weight of the topical composition (w/w), for example, about 0.0006% to about 0.75% w/w, about 0.0006% to about 0.5% w/w, about 0.0006% to about 0.25% w/w, about 0.0006% to about 0.1% w/w, about 0.0006% to about 0.05% w/w, about 0.0006% to about 0.01% w/w, about 0.0006% to about 0.005% w/w, about 0.0006% to about 0.001% w/w, about 0.001% to about 1.0% w/w, about 0.0025% to about 1.0% w/w, about 0.005% to about 1.0% w/w, about 0.01% to about 1.0% w/w, about 0.025% to about 1.0% w/w, about 0.05% to about 1.0% w/w, about 0.1% to about 1.0% w/w, about 0.25% to about 1.0% w/w, about 0.5% to about 1.0% w/w, about 0.75% to about 1.0% w/w, about 0.001% to about 0.5% w/w, about 0.0025% to about 0.5% w/w, about 0.005% to about 0.5% w/w, about 0.01% to about 0.5% w/w, about 0.025% to about 0.5% w/w, about 0.05% to about 0.5% w/w, about 0.1% to about 0.5% w/w, about 0.25% to about 0.5% w/w, about 0.001% to about 0.25% w/w, about 0.0025% to about 0.25% w/w, about 0.005% to about 0.25% w/w, about 0.01% to about 0.25% w/w, about 0.025% to about 0.25% w/w, about 0.05% to about 0.25% w/w, about 0.1% to about 0.25% w/w, about 0.001% to about 0.1% w/w, about 0.0025% to about 0.1% w/w, about 0.005% to about 0.1% w/w, about 0.01% to about 0.1% w/w, about 0.025% to about 0.1% w/w, about 0.05% to about 0.1% w/w, about 0.001% to about 0.05% w/w, about 0.0025% to about 0.05% w/w, about 0.005% to about 0.05% w/w, about 0.01% to about 0.05% w/w, about 0.025% to about 0.05% w/w, about 0.001% to about 0.025% w/w, about 0.0025% to about 0.025% w/w, about 0.005% to about 0.025% w/w, about 0.01% to about 0.025% w/w, or about 0.0125% to about 0.05% w/w of the topical composition.

In some examples, the topical composition includes the water extract of Chardonnay grape seed at about 0.006% to about 0.1% by weight of the topical composition (w/w). In some examples, the topical composition includes the water extract of Chardonnay grape seed at about 0.08% to about 0.05% w/w. In some examples, the topical composition includes the water extract of Chardonnay grape seed at about 0.01% to about 0.05% w/w. In some examples, the topical composition includes the water extract of Chardonnay grape seed at about 0.0125% to about 0.05% w/w.

In some examples, the topical composition includes the water extract of Chardonnay grape seed at about 0.0006%, about 0.00075%, about 0.001%, about 0.0025%, about 0.005%, about 0.0075%, about 0.01%, about 0.025%, about 0.05%, about 0.075%, about 0.1%, about 0.25%, about 0.5%, about 0.75%, or about 1.0% by weight of the topical composition (w/w). In some examples, the topical composition includes the water extract of Chardonnay grape seed at about 0.025% w/w. In a further example, the topical composition includes the water extract of Chardonnay grape seed at about 0.0125% w/w.

iii. Resveratrol

In some aspects, the topical composition includes resveratrol at about 0.001% to about 3.0% by weight of the topical composition (w/w), for example, about 0.0018% to about 2.5% w/w, about 0.0018% to about 2.0% w/w, about 0.0018% to about 1.5% w/w, about 0.0018% to about 1.0% w/w. about 0.0018% to about 0.5% w/w, about 0.0018% to about 0.25% w/w, about 0.0018% to about 0.1% w/w, about 0.0018% to about 0.05% w/w, about 0.0018% to about 0.025% w/w, about 0.0018% to about 0.01% w/w, about 0.002% to about 3.0% w/w, about 0.005% to about 3.0% w/w, about 0.01% to about 3.0% w/w, about 0.025% to about 3.0% w/w, about 0.05% to about 3.0% w/w, about 0.075% to about 3.0% w/w, about 0.1% to about 3.0% w/w, about 0.25% to about 3.0% w/w, about 0.5% to about 3.0% w/w, about 0.75% to about 3.0% w/w, about 1.0% to about 3.0% w/w, about 1.5% to about 3.0% w/w, about 2.0% to about 3.0% w/w, about 2.5% to about 3.0% w/w, about 0.0025% to about 2.0% w/w, about 0.005% to about 2.0% w/w, about 0.01% to about 2.0% w/w, about 0.025% to about 2.0% w/w, about 0.05% to about 2.0% w/w, about 0.1% to about 2.0% w/w, about 0.25% to about 2.0% w/w, about 0.5% to about 2.0% w/w, about 1.0% to about 2.0% w/w, about 1.5% to about 2.0% w/w, about 0.0025% to about 1.5% w/w, about 0.005% to about 1.5% w/w, about 0.01% to about 1.5% w/w, about 0.025% to about 1.5% w/w, about 0.0375% to about 1.5% w/w, about 0.05% to about 1.5% w/w, about 0.1% to about 1.5% w/w, about 0.375% to about 1.5% w/w, about 0.5% to about 1.5% w/w, about 1.0% to about 1.5% w/w, about 0.0025% to about 1.0% w/w, about 0.005% to about 1.0% w/w, about 0.01% to about 1.0% w/w, about 0.025% to about 1.0% w/w, about 0.0375% to about 1.0% w/w, about 0.05% to about 1.0% w/w, about 0.1% to about 1.0% w/w, about 0.375% to about 1.0% w/w, about 0.5% to about 1.0% w/w, about 0.0025% to about 0.5% w/w, about 0.005% to about 0.5% w/w, about 0.01% to about 0.5% w/w, about 0.025% to about 0.5% w/w, about 0.0375% to about 0.5% w/w, about 0.05% to about 0.5% w/w, about 0.1% to about 0.5% w/w, about 0.375% to about 0.5% w/w, about 0.0025% to about 0.15% w/w, about 0.005% to about 0.15% w/w, about 0.01% to about 0.15% w/w, about 0.025% to about 0.15% w/w, about 0.0375% to about 0.15% w/w, about 0.05% to about 0.15% w/w, about 0.1% to about 0.15% w/w, about 0.0025% to about 0.075% w/w, about 0.005% to about 0.075% w/w, about 0.01% to about 0.075% w/w, about 0.025% to about 0.075% w/w, about 0.0375% to about 0.075% w/w, about 0.05% to about 0.075% w/w, about 0.1% to about 0.075% w/w, or about 0.375% to about 0.75% w/w of the topical composition. The resveratrol can be trans-resveratrol. The resveratrol can be from a source other than Polygonum cuspidatum.

In some examples, the topical composition includes resveratrol at about 0.018% to about 0.3% by weight of the topical composition (w/w). In some examples, the topical composition includes resveratrol at about 0.025% to about 0.3% by weight of the topical composition (w/w). In some examples, the topical composition includes resveratrol at about 0.025% to about 0.15% by weight of the topical composition (w/w). In some examples, the topical composition includes resveratrol at about 0.0375% to about 0.15% w/w. The resveratrol can be trans-resveratrol. The resveratrol can be from a source other than Polygonum cuspidatum.

In some examples, the topical composition includes resveratrol at about 0.001%, about 0.00375%, about 0.005%, about 0.0075%, about 0.01%, about 0.0375%, about 0.05%, about 0.075%, about 0.1%, about 0.375%, about 0.5%, about 0.75%, about 1.0%, about 1.25%, about 1.5%, about 1.75%, about 2.0%, about 2.25%, about 2.5%, about 2.75%, or about 3.0% by weight of the topical composition (w/w). In some examples, the topical composition includes resveratrol at about 0.075% w/w. In a further example, the topical composition includes the resveratrol at about 0.0375% w/w. The resveratrol can be trans-resveratrol. The resveratrol can be from a source other than Polygonum cuspidatum.

In some examples, the resveratrol is substantially in the trans molecular configuration (trans-resveratrol). In some examples, the resveratrol is at least 50% trans-resveratrol, for example, at least 55%, at least 60%, at least 65%, at least 70%, at least 75%, at least 80%, at least 85%, at least 90%, at least 95%, at least 98%, at least 99%, or more trans-resveratrol. In a specific, non-limiting example, the resveratrol is at least 70% trans-resveratrol. In a further example, the resveratrol is at least 90% trans-resveratrol. In other examples, the resveratrol is at least 98% trans-resveratrol. In some examples, the resveratrol contains less than 50% cis-resveratrol or piceatannol, for example, less than 45%, less than 40%, less than 35%, less than 30%, less than 25%, less than 20%, less than 15%, less than 10%, less than 5%, less than 2.5%, less than 1%, less than 0.5%, or less than 0.1% cis-resveratrol or piceatannol of the total resveratrol. In some examples, the resveratrol contains less than 0.5% cis-resveratrol or piceatannol. The resveratrol can be trans-resveratrol. The resveratrol can be from a source other than Polygonum cuspidatum.

iv. Further Description of the Topical Composition

In some aspects, the topical composition includes the decolorized muscadine grape pomace extract, the water extract of Chardonnay grape seed, and resveratrol at a ratio of 1-10 parts the decolorized muscadine grape pomace extract, 1-5 parts the water extract of Chardonnay grape seed, and 2.5-7.5 parts resveratrol. In some examples, the topical composition includes 1-10 parts the decolorized muscadine grape pomace extract, 2.5 parts the water extract of Chardonnay grape seed, and 7.5 parts resveratrol. In some examples, the topical composition includes 10 parts the decolorized muscadine grape pomace extract, 2.5 parts the water extract of Chardonnay grape seed, and 7.5 parts resveratrol. In a further example, the topical composition includes 1 part the decolorized muscadine grape pomace extract, 2.5 parts the water extract of Chardonnay grape seed, and 7.5 parts resveratrol.

In some aspects, the topical composition includes the decolorized muscadine grape pomace extract at about 0.025% to about 0.4% w/w, the water extract of Chardonnay grape seed at about 0.006% to about 0.1% w/w, and resveratrol at about 0.018% to about 0.3% w/w. In some examples, the decolorized muscadine grape pomace extract at about 0.05% to about 0.2% w/w, the water extract of Chardonnay grape seed at about 0.0125% to about 0.05% w/w, and resveratrol at about 0.0375% to about 0.15% w/w. In some examples, the topical composition includes the decolorized muscadine grape extract at about 0.10% w/w, the water extract of Chardonnay grape seed at about 0.025% w/w, and resveratrol at about 0.075% w/w. In another example, the topical composition includes the decolorized muscadine grape extract at about 0.05% w/w, the water extract of Chardonnay grape seed at about 0.0125% w/w, and resveratrol at about 0.0375% w/w. In some examples, the topical composition including the decolorized muscadine grape extract at about 0.10% w/w, the water extract of Chardonnay grape seed at about 0.025% w/w, and resveratrol at about 0.075% w/w is a serum.

In some aspects, the topical composition includes the decolorized muscadine grape pomace extract at about 0.0025% to about 0.04% w/w, the water extract of Chardonnay grape seed at about 0.006% to about 0.1% w/w, and resveratrol at about 0.018% to about 0.3% w/w. In some examples, the topical composition includes the decolorized muscadine grape pomace extract at about 0.005% to about 0.02% w/w, the water extract of Chardonnay grape seed at about 0.0125% to about 0.05% w/w, and resveratrol at about 0.0375% to about 0.15% w/w. In another example, the topical composition includes the decolorized muscadine grape pomace extract at about 0.01% w/w, the water extract of Chardonnay grape seed at about 0.025% w/w, and resveratrol at about 0.075% w/w. In a further example, the topical composition includes the decolorized muscadine grape pomace extract at about 0.005% w/w, the water extract of Chardonnay grape seed at about 0.0125% w/w, and resveratrol at about 0.0375% w/w. In some examples, the topical composition including the decolorized muscadine grape pomace extract at about 0.01% w/w, the water extract of Chardonnay grape seed at about 0.025% w/w, and resveratrol at about 0.075% w/w is a lotion.

The topical composition can include additional ingredients, such as one or more vitamins, additional botanical extracts, phytochemicals, fats or oils, prebiotics, probiotics, enzymes, surfactants, emollients, emulsifiers, humectants, moisturizing agents, pH adjusters, skin conditioners, thickening agents, preservatives, sunscreen agents (UV absorbers; physical and chemical absorbers such as avobenzone, titanium dioxide, zinc oxide, etc.), skin bleaching or lightening agents, dyes, adsorbents, lubricants, solvents, water-repellants, anti-irritants, essential oils, anti-microbial agents, antioxidants, chelating agents, exfoliants, fragrances, or other desirable ingredients. Suitable additional ingredients have been described and can be selected by a formulator.

A vitamin is an organic molecule that is an essential micronutrient that an organism needs in small quantities for the proper functioning of its metabolism. Sources of vitamins are plant and animal food products and dietary supplements. Non-limiting examples of vitamins include Vitamin A (retinol and its analogues, such as retinyl palmitate), any of the 12 B-complex vitamins (e.g., thiamine (B1), riboflavin (B2), niacin or niacinamide (B3), pantothenic acid (B5), B6, biotin (B7), folate (B9), B12, and their analogs), Vitamin C (and its analogues, such as magnesium ascorbyl phosphate, ascorbyl palmitate, etc.), Vitamin D (e.g., calcitriol (D3)), Vitamin E (tocopherol and its analogues, such as tocopheryl acetate), Vitamin F (alpha-linolenic acid and linoleic acid), and Vitamin K (e.g., phylloquinone, menaquinones, and their analogs). In one example, the vitamin is Vitamin K. In another example, the vitamin is Vitamin F. In some examples, the topical composition includes a vitamin.

Botanical extracts are extracts derived from plants. Extracts can be made from any part of a plant, for example, from the leaf, root, stem, bark, flower, seed, kernel, fruit, or combinations thereof. Non-limiting examples of botanical extracts include, for example, extracts from Arnica montanais (commonly known a wolf's bane, leopard's bane, mountain tobacco, and mountain arnica), Symphytum (comfrey), Sesamum indicum (sesame), soy, tea (green, black, or white), Matricaria recurita (chamomile), Cofea arabica (coffee or coffeeberry), almond, angelica root, acacia, aloe, althea anise, apple, avocado, basil, bayberry, bilberry, blue malva flower, blackberry, burdock, borage, bergamot, buchu, cannabis, cranberry, clover blossom, calendula, clove, carrot, China bark, cinnamon, clary sage, coltsfoot, cornflower, cranesbill, cucumber, dandelion, dulse, eucalyptus, echinacea, elderberry, fennel, fenugreek, garlic, gentian root, geranium, ginko biloba, ginseng, grapefruit, grapeseed, goldenseal, hops, hawthorn, henna, horse chestnut, horsetail, hibiscus, ivy, Irish moss, jasmine, juniper berry, kelp, kiwi, linden flower, lovage, lavender, lemongrass, lemon peel, lemon balm, myrrh, mallow, meadow sweet (mayflower), melon, mint, melilot (hayflower), milk thistle, nettle, oatmeal, oat kernel, orange flower, orange peel, oak moss, peach, plantain, parsley, peppermint, primrose, papaya, quassia, quince, rose bud, rose hips, rosemary, sage, Sambucus elder, slippery elm bark, spearmint, Sambucus, sandalwood, southernwood, strawberry, sunflower, St. Johns wort, tangerine, tansy, thyme, tomato, uva ursi (bearbenry), violet, white lily, valerian root, watercress, walnut (black), white birch, white willow bark, wild indigo, witch hazel, yucca, yarrow, Magnolia Officinalis (Houpu Magnolia) bark, Prunus domestica (Plum) seed, Cocos nucifera (Coconut), Vitis vinifera (Chardonnay grape), Vitis rotundifolia (muscadine grape), Polygonum cuspidatum root, or other botanical source. In some examples, the topical composition includes a botanical extract in addition to the decolorized muscadine grape pomace extract or water extract of Chardonnay grape seed.

The topical composition can include synthetic or naturally derived phytochemicals, such as sesquiterpene lactones, rosmarinic acid, sesamin, sesamol, sesaminol, sesamolin, isoflavones (e.g., genistein and daidzein), polyphenolics (e.g., resveratrol, chlorogenic acid, ferulic acid, quinic acid, and condensed proanthocyanidins), epigallocatechin-3-gallate (EGCG), flavonoids (e.g., quercetin, theaflavin, kaempferol, apigenin, luteolin, rutin), catechins, terpenoids (e.g., bisoprolol, matricine, levomenol, chamazulene), caffeine, hydrolysable tannins, anthocyanidins, anthocyanosides, or combinations thereof.

In some examples, the topical composition includes a fat or oil. Non-limiting examples of fats or oils include coconut oil, argan oil, rosehip seed oil, marula oil, jojoba oil, black seed oil, sunflower seed oil, safflower oil, cacay oil, rosehip oil, other nut or seed oils, cocoa (cacao) butter, cocoglycerides, shea butter, mango butter, kokum butter, shorea butter, cupuacu butter, illipe butter, dicaprylyl carbonate, ceramides, or lecithin. The topical composition can also include fatty acids, such as linoleic acid, oleic acid, palmitic acid, stearic acid, linolenic acid, behenic acid, arachidic acid, or gondoic acid.

Probiotics are live microorganisms that are intended to have health benefits when consumed or applied to the body. Exemplary probiotics include Lactobacillus species, for example, Lactobacillus acidophilus, Lactobacillus casei, Lactobacillus plantarum, and Lactobacillus brevis. In some examples, the probiotic is Lactobacillus acidophilus. In some examples, the topical composition includes a probiotic. In some examples, the topical composition includes a filtered formation product, such as AMTICIDE® Coconut, examples a product produced from Lactobacillus fermentation of Cocos nucifera fruit.

Prebiotics are plant fibers/carbon sources that support the growth of probiotics. Exemplary prebiotics include bran, beta-glucan, inulin, alpha-glucan oligosaccharide, xylitol, rhamnose, and fructooligosaccharides. In some examples, the prebiotic is a postbiotic extract with a prebiotic effect, for example, BIOME OLÉOACTIF®. One or more prebiotics can be included in a disclosed composition. Plant sugars, such as xylitol, rhamnose, and fructooligosaccharides can also be included in a topical composition.

An enzyme is a protein that act as biological catalyst to accelerate the of chemical reactions. Non-limiting examples of enzymes include coenzyme Q10, superoxide dismutase (SOD), proteolytic enzymes (e.g., actinidin, ficin, pepsin, bromelain, and papain), diacylglycerol acyltransferase (DGAT-1), lysyl and prolyl hydroxylase, and peroxidase enzymes. In some examples, the topical composition includes an enzyme.

Surfactants are amphiphilic molecules that have hydrophobic and hydrophilic parts. Surfactants tend to reduce the surface tension or interfacial tension between two liquids, which can increase the solubility of organic compounds. Non-limiting examples of surfactants include glyceryl stearate, polyglyceryl-3 diisostearate, ammonium lauryl sulfate, sodium lauryl sulfate, sodium laureth sulfate, decyl glucoside, coco-glucoside, cocamidopropyl, hydroxysultaine, sodium methyl cocoyl taurate, sodium methyltaurate, and lauryl glucoside. In some examples, the topical composition includes a surfactant.

An emollient is a substance that helps soothe, soften, and increase moisture levels, particularly in skin or mucous membranes. Non-limiting examples of emollients include cetyl palmitate, polyglyceryl-3 diisostearate, squalene, petrolatum, lanolin, mineral oil, Cocos nucifera (coconut) oil, Adansonia digitata (baobab) seed oil, propanediol, dicaprylyl carbonate, cocoglycerides, and dimethicone. In some examples, the topical composition includes an emollient.

An emulsifier is a substance that stabilizes the suspension of one liquid in another, for example, a mixture of oil and water. Non-limiting examples of emulsifiers include cetyl palmitate, lecithin, cetearyl alcohol, coconut acid, maltodextrin, and polyglyceryl-3 diisostearate. In some examples, the topical composition includes an emulsifier.

A humectant is s a hygroscopic substance used to retain moisture. Humectants are commonly used as a moisturizing agent in lotions and other cosmetics used for skin. Non-limiting examples of humectants include glycerin, gluconolactone, caprylyl glycol, calcium gluconate, sodium hyaluronate, sodium acetylated hyaluronate, sodium hyaluronate crosspolymer, hydrolyzed sodium hyaluronate, propanediol, ethylhexylglycerin, and propylene glycol. In some examples, the topical composition includes a humectant.

The topical composition can include preservatives. Preservatives are substances used to protect against decay, discoloration, or spoilage. Non-limiting examples of preservatives include benzoic acid, propanediol, potassium sorbate, phenoxyethanol, caprylyl glycol, hydroxyacetophenone, phenethyl alcohoL AMTICIDE® (coconut fruit extract+Lactobacillus), pentylene glycol, ethylhexylglycerin, and sodium benzoate.

The topical composition can include UV absorption agents (sunscreen agents). UV absorption agents include chemical and physical sunblocks. Non-limiting examples of chemical sunblocks that can be used include salicylates (homomethyl salicylate, benzyl salicylate, glycol salicylate, isopropyl benzyl salicylate, etc.), anthranilates, ethyl urocanate, homosalate, dibenzoyl methane derivatives (e.g., avobenzone), octocrylene, etc. Non-limiting examples of physical sunblocks include metal oxides (e.g., titanium dioxide and zinc oxide).

Suitable additional ingredients are further described in section B, “Methods of Preparing the Topical Composition.”

The topical composition can be formulated in any form suitable for application to a surface of the body (i.e., skin), such as a serum, lotion, cream, gel, ointment, paste, solution, suspension, spray, or the like. In some examples, the topical composition is a cosmetic. In some examples, the cosmetic is a skin care product.

In some examples, the topical composition is a serum. Serums generally contains higher concentrations of active ingredients and are applied directly to clean skin prior to application of a moisturizer, such as lotion. A serum can have a low viscosity and a higher proportion of water than oil. In some examples, the serum composition includes about 60% to about 95% water, such as about 60%, about 65%, about 70%, about 75%, about 80%, about 85%, about 90%, or about 95%, based upon the weight of the serum composition (w/w). In a non-limiting example, the serum composition is about 85% water, based upon the weight of the serum composition (w/w).

In some examples, the topical composition is a lotion. Lotions are preparations to be applied to the skin surface, generally without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions can be used for treating large body areas, because of the ease of applying a more fluid composition. It is generally necessary that the insoluble matter in a lotion be finely divided. Lotions typically contain suspending agents to produce better dispersions as well as compounds useful for localizing and holding the active agent in contact with the skin, e.g., methylcellulose, sodium carboxymethyl-cellulose, or the like. In some examples, the lotion composition includes about 50% to about 80% water, such as about 50%, about 55%, about 60%, about 65%, about 70%, about 73%, about 75%, or about 80%, based upon the weight of the lotion composition (w/w). In a non-limiting example, the lotion composition is about 73% water, based upon the weight of the serum composition (w/w).

In some examples, the topical composition is a cream. Creams are viscous liquids or semisolid emulsions, either oil-in-water or water-in-oil. Cream bases are often water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

In some examples, the topical composition is a gel. Gels are semisolid, suspension-type systems. Single-phase gels contain organic macromolecules distributed substantially uniformly throughout the carrier liquid, which is typically aqueous, but also may contain an alcohol or an oil. Useful “organic macromolecules,” specifically gelling agents, are crosslinked acrylic acid polymers such as the “carbomer” family of polymers, e.g., carboxypolyalkylenes that are commercially available as CARBOPOL®. Also of use are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

In some examples, the topical composition is an ointment. Ointments are semisolid preparations that are typically based on petrolatum or petroleum derivatives. The specific ointment base to be used can be selected by a practitioner based on desired characteristics, e.g., emollience or the like. An ointment base is generally inert, stable, non-irritating, and non-sensitizing. Ointment bases are grouped in four classes: oleaginous bases; emulsifiable bases; emulsion bases; and water-soluble bases (see, e.g., Remington: The Science and Practice of Pharmacy, 23rd Ed. (Academic Press), 2021 (ISBN 9780128200070)). Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxy stearin sulfate, anhydrous lanolin, and hydrophilic petrolatum. Emulsion ointment bases are either water-in-oil (W/O) emulsions or oil-in-water (O/W) emulsions, and include, for example, acetyl alcohol, glyceryl monostearate, lanolin, and stearic acid. Water-soluble ointment bases are prepared from polyethylene glycols of varying molecular weight.

In some examples, the topical composition is a paste. Pastes are generally semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

In some examples, the topical composition is a solution. Solutions are generally homogeneous mixtures prepared by dissolving one or more chemical substances (solutes) in a liquid such that the molecules of the dissolved substance are dispersed among those of the solvent. The solution may contain other cosmetically acceptable chemicals to buffer, stabilize and/or preserve the solute. Common examples of solvents used in preparing topical solutions are ethanol, water, propylene glycol or any other acceptable vehicles. These can be applied in any manner, such as spraying them on the skin, painting them on the skin, or wetting a bandage with the solution.

The topical composition can be formulated as a moisturizer. Moisturizers soften and increase water retention of the skin by forming an oily layer on the top of the skin to trap water in the skin. Moisturizers often include emollients, such as petrolatum, lanolin, mineral oil and dimethicone, and humectants, including glycerin, lecithin, and propylene glycol, which draw water into the outer layer of skin. Many moisturizing products also have ingredients that soften keratin, such as urea, alpha hydroxy acids such as lactic/citric/glycolic acid, and allantoin. Compounds, such as zinc oxide or white petrolatum, that protect the skin against irritation can also be included.

The topical composition can be formulated for application to any external part of the body. In some non-limiting examples, the topical composition is formulated for application to the head, face, scalp, arms, hands, legs, feet, or body. In some examples, the topical composition is formulated for application to the body or face. Application methods generally depend on viscosity of the composition to be applied. Lower viscosity compositions can be applied with a dropper, swab, sprayer, or the like.

B. Methods of Preparing the Topical Composition

Also disclosed are methods of preparing the topical compositions disclosed herein. The methods include providing a topical composition base and mixing with the base an amount of the decolorized muscadine grape pomace extract, the water extract of Chardonnay grape seed, and resveratrol, respectively, sufficient to achieve a desired concentration (such as a concentration of the decolorized muscadine grape pomace extract, the water extract of Chardonnay grape seed, and resveratrol, respectively, as disclosed herein). In one example, the method includes mixing with the base:
I) the decolorized muscadine grape pomace extract at either
   a) about 0.025% to about 0.4% by weight of the topical composition (w/w), or
   b) about 0.0025% to about 0.04% by weight of the topical composition (w/w),
II) the water extract of Chardonnay grape seed at about 0.006% to about 0.1% by weight of the topical composition (w/w), and
III) resveratrol at about 0.018% to about 0.3% by weight of the topical composition (w/w).

In some examples, the method includes mixing the decolorized muscadine grape pomace extract at about 0.025% to about 0.4% by weight of the topical composition (w/w). In some examples, the method includes mixing the decolorized muscadine grape pomace extract at about 0.05% to about 0.2% w/w. In some examples, the method includes mixing the decolorized muscadine grape pomace extract at about 0.10% w/w. In a further example, the method includes mixing the decolorized muscadine grape pomace extract at about 0.05% w/w.

In some examples, the method includes mixing the decolorized muscadine grape pomace extract at about 0.0025% to about 0.04% by weight of the topical composition (w/w). In some examples, the method includes mixing the decolorized muscadine grape pomace extract at about 0.005% to about 0.02% w/w. In some examples, the method includes mixing the decolorized muscadine grape pomace extract at about 0.01% w/w. In a further example, the method includes mixing the decolorized muscadine grape pomace extract at about 0.005% w/w.

In some examples, the method includes mixing the water extract of Chardonnay grape seed at about 0.006% to about 0.1% by weight of the topical composition (w/w). In some examples the method includes mixing the water extract of Chardonnay grape seed at about 0.0125% to about 0.05% w/w. In a further example, the method includes mixing the water extract of Chardonnay grape seed at about 0.025% w/w.

In some examples, the method includes mixing resveratrol at about 0.018% to about 0.3% by weight of the topical composition (w/w). In some examples, the method includes mixing resveratrol at about 0.0375% to about 0.15% w/w. In some examples, the method includes mixing resveratrol at about 0.075% w/w.

In some aspects, the methods include mixing with the topical composition base a disclosed ratio of the decolorized muscadine grape pomace extract, the water extract of Chardonnay grape seed, and resveratrol. In an example, the methods include mixing 1 to 10 parts of the decolorized muscadine grape pomace extract, 1 to 5 parts of the water extract of Chardonnay grape seed, and 5 to 7.5 parts of resveratrol into the topical composition base. In another example, the methods include mixing 1 to 10 parts of the decolorized muscadine grape pomace extract, 2.5 parts of the water extract of Chardonnay grape seed, and 7.5 parts of resveratrol into the topical composition base. In a non-limiting example, the methods include mixing 1 part of the decolorized muscadine grape pomace extract, 2.5 parts of the water extract of Chardonnay grape seed, and 7.5 parts of resveratrol into the topical composition base. In another non-limiting example, the methods include mixing 10 parts of the decolorized muscadine grape pomace extract, 2.5 parts of the water extract of Chardonnay grape seed, and 7.5 parts of resveratrol into the topical composition base.

The base is a carrier for the topical composition, such as a liquid, gel, foam, cream, ointment, or lotion. Particularly useful carriers are those that are cosmetically acceptable for topical applications, such as one or more aqueous systems, glycerin, C₁₄ alcohols, fatty alcohols, fatty ethers, fatty esters, polyols, glycols, vegetable oils, mineral oils, liposomes, laminar lipid materials, silicone oils, water, or combinations thereof. Suitable bases (or carriers) have been described, for example, in Remington: The Science and Practice of Pharmacy, 23rd Ed. (Academic Press), 2021 (ISBN 9780128200070). In some examples, the base is a serum or lotion.

The CTFA International Cosmetic Ingredient Dictionary and Handbook (2004 and 2008) further discloses a variety of cosmetic ingredients that can be used. Exemplary ingredients disclosed below can be added during formulation.

In some examples, fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11, and iron oxides), adsorbents, lubricants, solvents, moisturizers (including, e.g., emollients, film formers, and agents that affect the natural moisturizing mechanisms of the skin), water-repellants, UV absorbers (physical and chemical absorbers such as avobenzone, titanium dioxide, zinc oxide, etc.), essential oils, trace metals (e.g., zinc, calcium and selenium), anti-irritants (e.g., steroids and non-steroidal anti-inflammatories), botanical extracts (e.g., aloe vera, chamomile, cucumber extract, Ginkgo biloba, ginseng, and rosemary), anti-microbial agents, antioxidants (e.g., tocopherol), chelating agents (e.g., sodium phytate and trisodium ethylenediamine disuccinate), preservatives (e.g., phenyoxyethanol, caprylyl glycol and chlorphenesin), pH adjusters (e.g., sodium hydroxide and citric acid), absorbents (e.g., aluminum starch octenyl succinate, kaolin, corn starch, oat starch, cyclodextrin, and zeolite), skin bleaching and lightening agents (e.g., hydroquinone and niacinamide lactate), humectants (e.g., glycerol, sorbitol, urea, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycynrhizate) can be added during the formulation process. Non-limiting examples of some of these ingredients are provided below.

UV Absorption Agents or Sunscreens: Sunscreens can be included in the moisturizers disclosed herein. A sunscreen alone may be unable to completely protect the skin against the adverse effects of ultraviolet radiation. Any skin care component made with the compositions described herein may additionally include a sunscreen and/or a UV absorption agent. In some examples, at least one of the five components further comprises a sunscreen, such as a sunscreen of SPF about 15-50. In an example, the day moisturizer further comprises a sunscreen, which can have a SPF of about 20, about 25, about 30, about 35, about 40, about 45 or about 50.

UV absorption agents that can be used in combination with the compositions of the present disclosure include chemical and physical sunblocks. Non-limiting examples of chemical sunblocks that can be used include salicylates (homomethyl salicylate, benzyl salicylate, glycol salicylate, isopropyl benzyl salicylate, etc.), anthranilates, ethyl urocanate, homosalate, dibenzoyl methane derivatives (e.g., avobenzone), octocrylene, etc. Non-limiting examples of physical sunblocks include metal oxides (e.g., titanium dioxide and zinc oxide).

Moisturizing Agents: Moisturizing agents can be included in the cleanser, toner, serum, or moisturizers disclosed herein. Non-limiting examples of moisturizing agents that can be used with any of the compositions and components described herein include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, propanediol, polyglyceryl sorbitol, salts of pyrollidone carboxylic acid, potassium PCA, sodium glucuronate, sodium PCA, sorbitol, sucrose, trehalose, urea, and xylitol.

Other examples of a moisturizing agent include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (Borago officinalis) extract, butcherbroom (ruscus aculeatus) extract, butylene glycol, Calendula officinalis extract, Calendula officinalis oil, candelilla (euphorbia cerifera) wax, canola oil, caprylic/capric triglyceride, cardamon (elettaria cardamomum) oil, carnauba (copernicia cerifera) wax, carrot (Daucus carota sativa) oil, castor (Ricinus communis) oil, ceramides, ceresin, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (Theobroma cacao) butter, coco-caprylate/caprate, coconut (Cocos nucifera) oil, collagen, collagen amino acids, corn (Zea mays) oil, fatty acids, decyl glucose, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (Oenothera biennis) oil, fatty acids, geranium maculatum oil, glucosamine, glucose glutamate, glutamic acid, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed glycosaminoglycans, hydrolyzed soy protein, hydroxylated lanolin, hydroxyproline, isocetyl stearate, isocetyl stearoyl stearate, isodecyl oleate, isopropyl isostearate, isopropyl lanolate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, lavender (Lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methyl gluceth-20, methylsilanol PCA, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (citrus aurantium dulcis) oil, palm (Elaeis guineensis) oil, palmitic acid, pantethine, panthenol, panthenyl ethyl ether, PCA, peach (prunus persica) kernel oil, peanut (Arachis hypogaea) oil, pentadecalactone, peppermint (Mentha piperita) oil, phospholipids, polyamino sugar condensate, polyglyceryl-3 diisostearate, polyglyceryl-4 caprate, polyquaternium-24, polysorbate 20, polysorbate 40, polysorbate 60, polysorbate 80, polysorbate 85, potassium myristate, potassium palmitate, pyridoxine dipalmitate, retinol, retinyl palmitate, rice (Oryza sativa) bran oil, RNA, rosemary (Rosmarinus officinalis) oil, rose oil, safflower (carthamus tinctorius) oil, sage (salvia officinalis) oil, sandalwood (santalum album) oil, serine, serum protein, sesame (Sesamum indicum) oil, shea butter (butyrospermum parkii), sodium hyaluronate, sodium lactate, sodium palmitate, sodium PCA, sodium polyglutamate, sodium stearoyl lactylate, sorbitan laurate, sorbitan oleate, sorbitan olivate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sunflower seed wax, sweet almond (prunus amygdalus dulcis) oil, tocopherol, tocopheryl acetate, tocopheryl linoleate, tribehenin, tridecyl neopentanoate, tridecyl stearate, tristearin, urea, vegetable oil, water, waxes, wheat (Triticum vulgare) germ oil, and ylang ylang (cananga odorata) oil.

Structuring Agents: Any of the disclosed compositions can include a structuring agent. Structuring agents, in certain examples, assist in providing rheological characteristics to the composition to contribute to the composition's stability. In other examples, structuring agents can also function as an emulsifier or surfactant. Non-limiting examples of structuring agents include stearic acid, palmitic acid, and mixtures thereof.

Essential Oils: Essential oils can be included in any of the compositions disclosed herein. Essential oils include oils derived from herbs, flowers, trees, and other plants. Such oils are typically present as tiny droplets between the plant's cells and can be extracted by several method known to those of skill in the art (e.g., steam distilled, enfleurage (i.e., extraction by using fat), maceration, solvent extraction, or mechanical pressing). When these types of oils are exposed to air, they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to about 240° C. and densities ranging from about 0.759 to about 1.096 g/mL.

Essential oils typically are named by the plant from which the oil is found. For example, rose oil or peppermint oil are derived from rose or peppermint plants, respectively. Non-limiting examples of essential oils that can be used include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present disclosure.

Thickening Agents: Thickening agents can be included in any of the compositions disclosed herein. Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions and components described herein. In certain aspects of the present disclosure, thickeners include hydrogenated polyisobutene or trihydroxystearin, or a mixture of both.

Non-limiting examples of additional thickening agents that can be used in the context of the present disclosure include carboxylic acid polymers, crosslinked polyacrylate polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich). Non-limiting examples of crosslinked polyacrylate polymers include cationic and nonionic polymers. In an example, the thickener includes an acrylate/C₁₀-C₃₀ alkyl acrylate crosspolymer. In certain examples, the thickener includes a copolymer of hydroxyethyl acrylate and sodium acryloyldimethyl taurate. Examples are described in U.S. Pat. Nos. 5,100,660; 4,849,484; 4,835,206; 4,628,078; 4,599,379).

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically, these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every third unit.

Non-limiting examples of gums that can be used with the compositions and components described herein, include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, sclerotium gum, sodium carboyxmethyl dextran, sodium carrageenan, tragacanth gum, xanthan gum, and mixtures thereof.

Preservatives: Any of the disclosed components can include a preservative. Non-limiting examples of preservatives that can be used in the context of the present disclosure include phenoxyethanol, chlorphenesin, caprylyl glycol, or combinations thereof.

Skin Lightening Agents: Any of the disclosed components can include a skin lightening agent. Non-limiting examples of skin lightening agents that can be used in the context of the present disclosure include dipotassium glycynrhizate, ascorbyl glucoside, niacinamide, licorice extract, or combination thereof.

Pharmaceutical Ingredients: Any of the disclosed components can include pharmaceutically active agents, although in some examples the disclosed components do not include pharmaceutically active agents. Non-limiting examples of pharmaceutical active agents include anti-acne agents, agents used to treat rosacea, analgesics, anesthetics, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, wart treatment agents, wound treatment agents, and wound healing agents.

In particular non-limiting examples, the methods include adding additional ingredients during formulation of the topical composition, such as one or more vitamins, additional botanical extracts, fats or oils, surfactants, emollients, emulsifiers, humectants, moisturizers, prebiotics, probiotics, enzymes, or other ingredient disclosed herein. In some non-limiting examples, the methods include adding prebiotics and/or postbiotics. In further non-limiting examples, the methods include adding alpha-glucan oligosaccharide, and a plant sugar, such as xylitol, rhamnose, and fructooligosaccharides. In further examples, the methods include adding Simmondsia Chinensis Seed Oil and/or Avena sativa Kernel Oil. In some examples, the methods include adding BIOME OLÉOACTIF®.

Additional ingredients can be added before, after, or substantially simultaneously with the decolorized muscadine grape extract, the water extract of Chardonnay grape seed, and resveratrol. In some examples, the additional components are added after the decolorized muscadine grape extract, the water extract of Chardonnay grape seed, and resveratrol have been mixed with the topical composition base. In some examples, the additional components are added before the decolorized muscadine grape extract, the water extract of Chardonnay grape seed, and resveratrol are mixed with the topical composition base.

In some examples, the topical composition is a serum, and the methods include formulating the serum. A serum composition can, for example, include water, moisturizing agents, pH adjusters, skin conditioners, thickening agents, surfactants, preservatives, and fragrances.

In some examples, the topical composition is a lotion, and the methods include formulating the lotion. A lotion is a moisturizer applied to the skin to add moisture to the external layers of the skin. In some examples, the lotion is formulated as a day moisturizer, night moisturizer, or all-purpose moisturizer (to be used day or night).

Moisturizers can act on the skin in up to three general ways: by forming a film on the surface of the skin to reduce loss of moisture already in the skin, by attracting water vapor from the air to increase moisture in the skin, or by adding moisturizing factors to the skin surface. A moisturizer composition can be in the form of a liquid, gel or semi-solid. In an example, the moisturizer composition is formulated as an emulsion. Skin moisturizers can make the skin softer and more flexible. The moisturizer composition can include a humectant, lipids, oils, surfactants, and pharmaceutically acceptable carriers. A humectant can increase the moisture level of the skin by both reducing water loss and by attracting ambient moisture to the skin. Glycerin, urea, and collagen are humectants that can be incorporated into a moisturizer composition.

A day moisturizer contains a sunscreen. In some examples, the day moisturizer composition contains a sunscreen of SPF about 15 to about 50, such as an SPF of about 15, about 20, about 25, about 30, about 35, about 40, about 45 or about 50. In certain non-limiting examples, the day moisturizer composition contains a sunscreen of SPF about 50. In certain examples, the day moisturizer composition includes water, moisturizing agents, sunscreens, skin conditioners, thickening agents, preservatives, and fragrances.

A night moisturizer is applied to the skin to provide a barrier to protect the skin surface, can act as a vehicle for topical delivery of additional ingredients to the skin, but does not contain a chemical sunscreen. A night moisturizer composition can be in the form of a liquid, gel or semi-solid. In an example, the night moisturizer composition is formulated as a semi-solid emulsion of oil and water. A night moisturizer composition differs from a day moisturizer component, as described herein, in that a night moisturizer composition does not provide sun protection, such as by containing a sunscreen, and is designed to infuse anti-aging ingredients, such as Vitamin A, deep within the skin's surface. Moisturizer compositions, including night moisturizer compositions, can make the skin softer and form a barrier to retain moisture in the skin. The night moisturizer composition can include a humectant, lipids, oils, surfactants, and pharmaceutically acceptable carriers. Use of a night moisturizer composition can counteract skin dryness due to frequent and/or harsh cleaning. Night moisturizer compositions may also contain glycerol, emulsifying agents, and preservatives.

C. Methods of Use

Disclosed herein are methods of inhibiting elastase or matrix metalloproteinase-1 (MMP-1), decreasing advanced glycation end product (AGE) formation or decreasing AGE content of collagen, or decreasing collagen breakdown in skin of a subject. The methods include applying to the skin of a subject, an effective amount of the topical composition disclosed herein. In some examples, the effective amount of the topical composition is an amount that inhibits elastase or MMP-1, decreases AGE formation or AGE content of collagen, or decreases collagen breakdown in the skin of the subject.

In some examples, the topical composition inhibits elastase or matrix metalloproteinase-1 (MMP-1) in the skin of a subject. Inhibiting includes decreasing enzymatic activity of elastase or MMP-1. In some examples, elastase or MMP-1 are inhibited by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more relative to a suitable control (e.g., activity prior to treatment or relative to an untreated control). In some examples, elastase or MMP-1 are inhibited by at least 25%, for example at least 26%, in the skin of the subject.

In some examples, elastase and/or MMP-1 are inhibited by about 5% to 100%, for example, by about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 80%, about 40% to about 100%, about 50% to about 75%, about 50% to about 100%, about 60% to about 100%, about 70% to about 100%, about 75% to about 100%, about 85% to about 100%, about 90% to about 100%, or about 95% to about 100% relative to a suitable control (e.g., activity prior to treatment or relative to an untreated control). In a non-limiting example, elastase and/or MMP-1 are inhibited by about 30% to about 70% relative to a suitable control. In another non-limiting example, elastase and/or MMP-1 are inhibited by about 48% to about 60% relative to a suitable control.

In some examples, the muscadine grape pomace extract, the water extract of Chardonnay grape seed, and resveratrol act synergistically in the topical composition. In some examples, the topical composition inhibits elastase and/or MMP-1, and for example, the amount of inhibition is greater than an additive effect of the decolorized muscadine grape pomace extract, the water extract of Chardonnay grape seed, and resveratrol individually.

In some examples, the topical composition decreases AGE formation or AGE content of collagen. In some examples, AGE formation or AGE content of collagen is decreased by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more relative to a suitable control (e.g., content prior to treatment or relative to an untreated control). In some examples, AGE formation or AGE content of collagen is decreased by at least 30% as compared to a suitable control (e.g., activity prior to treatment or relative to an untreated control). In some examples, the topical composition inhibits AGE formation and/or AGE content, and for example, the amount of inhibition is greater than the expected additive effect of the decolorized muscadine grape pomace extract, the water extract of Chardonnay grape seed, and resveratrol individually.

In some examples, AGE formation or AGE content of collagen is decreased by about 5% to 100%, for example, by about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 80%, about 40% to about 100%, about 50% to about 75%, about 50% to about 100%, about 60% to about 100%, about 70% to about 100%, about 75% to about 100%, about 85% to about 100%, about 90% to about 100%, or about 95% to about 100% relative to a suitable control (e.g., content prior to treatment or relative to an untreated control). In a non-limiting example, AGE formation or AGE content of collagen is decreased by about 30% to about 40% relative to a suitable control. In another non-limiting example, AGE formation or AGE content of collagen is decreased by about 30% to about 34% relative to a suitable control. In a further non-limiting example, AGE formation or AGE content of collagen is decreased by about 30.5% to about 33.5% relative to a suitable control.

In some examples, the topical composition synergistically decreases AGE formation or AGE content of collagen, for example, the amount of inhibition is greater than an additive effect of the decolorized muscadine grape pomace extract, the water extract of Chardonnay grape seed, and resveratrol individually.

Also disclosed are methods of improving the appearance of skin, including applying to the skin of a subject an effective amount of the topical composition disclosed herein. In some examples, the topical composition is applied for a period of time sufficient to improve the appearance of skin. In some examples, the effective amount is an amount that increases skin firmness, increases skin elasticity, or increases skin hydration relative to a suitable control (e.g., measurements prior to treatment or relative to an untreated control). In some examples, skin firmness, elasticity, or hydration is increased by at least 5%, for example, at least 7%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 250%, at least 300%, at least 400%, at least 500%, or more, relative to a suitable control (e.g., measurements or observations prior to treatment or relative to an untreated control).

In some examples, skin elasticity, firmness, or hydration is increased by about 5% to 500%, for example, by about 10% to about 500%, about 20% to about 500%, about 30% to about 500%, about 40% to about 500%, about 50% to about 500%, about 60% to about 500%, about 70% to about 500%, about 80% to about 500%, about 90% to about 500%, about 100% to about 500%, about 110% to about 500%, about 120% to about 500%, about 130% to about 500%, about 140% to about 500%, about 150% to about 500%, about 160% to about 500%, about 170% to about 500%, about 180% to about 500%, about 190% to about 500%, about 200% to about 500%, about 250% to about 500%, about 300% to about 500%, about 350% to about 500%, about 400% to about 500%, about 450% to about 500%, about 10% to about 450%, about 10% to about 400%, about 10% to about 350%, about 10% to about 300%, about 10% to about 250%, about 10% to about 200%, about 10% to about 190%, about 10% to about 180%, about 10% to about 170%, about 10% to about 160%, about 10% to about 150%, about 10% to about 140%, about 10% to about 130%, about 10% to about 120%, about 10% to about 110%, about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 5% to about 50%, about 5% to about 100%, about 10% to about 200%, about 25% to about 50%, about 25% to about 100%, about 25% to about 150%, about 25% to about 200%, about 25% to about 250%, about 25% to about 300%, about 50% to about 100%, about 50% to about 150%, about 50% to about 200%, about 50% to about 250%, about 50% to about 300%, about 100% to about 150%, about 100% to about 200%, about 100% to about 250%, about 100% to about 300%, about 100% to about 400%, about 100% to about 500%, about 250% to about 500%, relative to a suitable control (e.g., measurements or observations prior to treatment or relative to an untreated control).

Increasing skin elasticity, firmness, or hydration can be measured subjectively (qualitative) or objectively (quantitative). In some examples, an increase is based on a subjective measurement (e.g., self-assessment/perception by the subject). In some examples, an increase is based on an objective measurement (e.g., experimental or clinical data measuring of firmness, elasticity, or hydration).

In some examples, the topical composition is a serum, and includes 0.10% w/w, the water extract of Chardonnay grape seed at about 0.025% w/w, and resveratrol at about 0.075% w/w. In other examples, the topical composition is a lotion, and includes decolorized muscadine grape pomace extract at about 0.01% w/w, the water extract of Chardonnay grape seed at about 0.025% w/w, and resveratrol at about 0.075% w/w.

The subject can be any mammalian subject, including human or non-human mammals. In some examples, the subject is a human. The subject can be a child or an adult, for example, a child under the age of 18 or an adult age 18 or older. In some examples, the adult is over about 18, over about 25, over about 30, over about 35, over about 40, over about 45, over about 50, over about 55, over about 60, over about 65, over about 70, or over about 75 years of age. In further examples, the adult is about 18 to about 25, about 18 to about 35, about 18 to about 65, about 20 to about 35, about 25 to about 65, about 35 to about 65, about 40 to about 65, about 40 to about 80, about 65 to about 80, or over about 80. In some examples, the subject is a female adult over the age of 30. In some examples, the subject is a female adult over the age of 40.

The effective amount and application regimen can depend on the subject being treated (e.g., human, or non-human mammal), age, and general condition of the subject, among other factors. Thus, an “effective amount” of the topical composition will fall in a relatively broad range that can be determined by a practitioner through testing and trials.

The topical composition may be applied as a single application or multiple applications. In some examples, the topical composition is applied as needed. In some examples, the topical composition is applied, for example, twice weekly, three times weekly, four times weekly, five times weekly, once daily, twice daily, three times daily, four times daily, or more. The topical composition can be applied over a course of hours, days, months, or years. In some examples, the topical composition is applied for about 7 days, about 14 days, about 30 days, about 60 days, about 90 days, about 120 days, or longer. In some examples, the topical composition is applied for about one week, about two weeks, about three weeks, about four weeks, about six weeks, about eight weeks, about twelve weeks, or longer. In some examples, the topical composition is applied for about one month, about two months, about three months, about four months, about six months, about eight months, about twelve months, about 1.5 years, about 2 years, about 2.5 years, about 3 years, about 3.5 years, about 4 years, about 5 years, or longer. In a non-limiting example, the topical composition is applied at least once a day for at least 30 days. In another example, the topical composition is applied at least twice a day for at least 30 days. In a further example, the topical composition is applied at least once a day for at least 60 days.

In some examples, applying the topical composition includes applying a serum (as disclosed herein) followed by applying a lotion (as disclosed herein). In a non-limiting example, the serum includes the decolorized muscadine grape extract at about 0.10% w/w, the water extract of Chardonnay grape seed at about 0.025% w/w, and resveratrol at about 0.075% w/w; and the lotion includes the decolorized muscadine grape pomace extract at about 0.01% w/w, the water extract of Chardonnay grape seed at about 0.025% w/w, and resveratrol at about 0.075% w/w.

### Examples

### Example 1

Composition of Botanical Extract Blends Extract Blend A (also referred to as test blend 18) was prepared by mixing the decolorized muscadine grape pomace extract, the water extract of Chardonnay grape seed, and resveratrol at a ratio of 10:2.5:7.5 (Muscadine Grape/Chardonnay Grape/Resveratrol). Extract Blend B (also referred to as test blend 22) was prepared by mixing the decolorized muscadine grape pomace extract, the water extract of Chardonnay grape seed, and resveratrol in a ratio of 1:2.5:7.5 (Muscadine Grape/Chardonnay Grape/Resveratrol). Table 7 provides the nutritional constituents of Extract Blend A and Extract Blend B. Table 8 shows polyphenol constituents (and analysis method) for Extract Blend A and Extract Blend B.

TABLE 7
Nutritional constituents of extract blends (% dry basis)

| | Extract Blend A | Extract Blend B |
|---|---|---|
| | Ratio 10:2.5:7.5 | Ratio 1:2.5:7.5 |
| | (50%/12.5%/37.5%) | (9.1%/22.7%/68.2%) |
| | (RM113/ | (RM113/ |
| | YG093/YR066) | YG093/YR066) |
| Nutritional Class | (% Dry Basis) | (% Dry Basis) |
| Polyphenols (GAE) | 45% (Average) | 76% (Average) |
| Fiber | 1.5 | 2.7 |
| Protein | 1.2 | 1.5 |
| Fat | <0.5 | <0.5 |
| Sugars | 5.5 | 7.0 |
| Sugar Alcohols | 4.4 | 0.8 |
| Organic Acids | 8.0 | 1.5 |
| Ash (Inorganic | 9.5 | 1.8 |
| Constituents) | | |
| Sugar Acids | 8.3 | 1.5 |
| Amino Acids | 3.9 | 0.7 |
| Moisture | 8.0 | 3.7 |
| “other” | 4.2 | 2.3 |
| Total | 100.0 | 100.0 |

TABLE 8
Polyphenol constituents of extract blends A and B (% dry basis)

| | Extract | Extract | |
|---|---|---|---|
| | Blend A | Blend B | |
| | Ratio | Ratio | |
| | 10:2.5:7.5 | 1:2.5:7.5 | |
| | (% Dry | (% Dry | |
| Polyphenol Class | Basis) | Basis) | Analysis Method |
| Catechins | 3.4 | 0.30 | HPLC/MS Quantitation |
| | | | (280 nm) |
| | | | (+/−)-Catechin Std |
| Procyanidins (n = 2-3 | 10.6 | 19.3 | HPLC/MS Quantitation |
| flavanol-3-ol units) | | | after thiolysis |
| Proanthocyanidins: | 13.8 | 5.7 | n-Butanol-HCL assay |
| Oligomeric/Polymeric | | | |
| (n = 4-10 flavanol-3-ol | | | |
| units) | | | |
| Ellagic Acid | 1.3 | 0.20 | HPLC/MS Quantitation w/ |
| Ellagic Acid Glycosides | 1.6 | 0.20 | Respective Standards |
| Gallic Acid | 15.4 | 2.8 | |
| Quercetin/Glycosides | 1.3 | 0.20 | |
| Gallotannins | 5.0 | 0.90 | Hydrochloric Acid |
| | | | Hydrolysis HPLC/MS |
| Ellagitannins | 3.9 | 0.70 | Quantitation: Ellagic Acid |
| | | | and Methyl Gallate |
| | | | Increase |
| Anthocyanins | 2.3 | 0.42 | HPLC/MS Quantitation |
| | | | (520 nm) |
| | | | Cyanidin-3,5-Diglucoside |
| | | | Std |
| Phenolic Acids | 3.0 | 0.88 | HPLC/MS Quantitation |
| | | | (254 nm) |
| | | | Chlorogenic Acid |
| Stilbenes | 37 | 67 | HPLC/MS Quantitation |
| | | | (306 nm) |
| | | | trans-Resveratrol |
| “Other” | 1.40 | 1.40 | n/a |
| Total | 100.0 | 100.0 | |

### Example 2

### Bioassay Materials and Methods

Botanical extract Blend A and Blend B were tested for activity in a variety of bioassays aimed to evaluate enhanced skin care benefits, as outlined in Table 9 below:

TABLE 9
in vitro bioassay testing performed

| Measurement | Benefit Area | Cells/Methods Used |
|---|---|---|
| Elastase | Elasticity | Human Dermal |
| | | Fibroblast Cell |
| | | Cultures/Spectrophotometric |
| | | (405 nm) |
| Collagenase | Firmness | Non-Cell/Spectrophotometric |
| (MMP-1) | | (412 nm) |
| AGE (advanced | Antiaging (Reduction | Non-Cell/Spectrophotometric |
| glycation end | and Prevention | (355 nm & 405 nm) |
| | of Collagen | |
| products) | Breakdown) | |
| DPPH | Antioxidant | Non-Cell/Spectrophotometric |
| | | (490 nm) |
| Cyclobutane | Prevention of | MatTek Epiderm (Epi-200) |
| Pyrimidine Dimer | DNA damage | Tissue |

Various bioassays demonstrated skin health benefits and, in some instances, unexpected, enhanced benefits from Botanical Extract Blends A and B. The tests provided in Table 9 were performed as follows:

a. Elastase and Collagenase Inhibition

Elastase

Human dermal fibroblasts were cultured and used as a source of the elastase enzyme. Elastase was partially purified from fibroblasts by lysing the cells in an elastase buffer and retaining the soluble portion of the lysate. Portions of this fibroblast lysate were then be incubated with test materials and a synthetic elastase substrate, Suc-(Ala3)-p-Nitroaniline (STANA). Elastase acts upon the substrate, releasing p-nitroaniline, which can be detected with a spectrophotometer by measuring absorbance at a wavelength of 405 nm. Inhibition of elastase enzyme is indicated by a decrease in the amount of released p-nitroaniline as compared to the uninhibited enzyme.

Collagenase

Matrix Metalloproteinase-1 (MMP-1) is an extracellular protease with an approximate molecular weight of 52-56 kD in its latent form. Upon cleavage of the proenzyme, the 22-46 kD MMP-1 becomes an active enzyme and can degrade many substrates including collagen, gelatin, and entactin. In human skin, increased MMP-1 activity can be induced via some disease states, exposure to UV irradiation or as part of the natural aging process. This can result in an imbalanced state where the degradation of collagen by MMP-1 exceeds its rate of replacement. Therefore, materials that inhibit MMP-1 activity can be beneficial for skin care. To screen inhibitors of MMP-1, human recombinant MMP-1 was incubated in the presence of a thiopeptolide substrate, potential inhibitors, and 5,5′-dithiobis(2-nitrobenzoic acid) (DTNB). When the thiopeptolide substrate is broken down by MMP-1, it releases a sulfhydryl group that can react with DTNB and form 2-nitro-5-thiobenzoic acid, which can be detected spectrophotometrically at 412 nm. Thus, in the presence of inhibitors, absorbance will be decrease relative to the non-inhibited enzyme.

b. AGE Assay (Prevention and Reduction of Collagen Breakdown)

AGE Prevention—50 μl of ice-cold collagen solution was added to the wells of a 96 well plate. The plate was incubated overnight at 37° C. to allow the collagen solution to gel. On the following day, 40 μl of either test material (prepared in assay buffer), aminoguanidine (positive control), or assay buffer (negative control) was added to the plate. After the materials were added, 10 μl of a 500 mM glyceraldehyde solution was added to all of the wells. The plate was then read using a fluorometer (355 nm excitation and 405 nm emission) and then incubated for 24 hours in a humidified incubator at 37C. On the following day the plate was read again. AGE formation was determined by subtracting the final fluorescent measurement from the initial fluorescent measurement for each sample.

AGE Reduction—50 μl of ice-cold collagen solution was added to the wells of a 96 well plate. The plate was then incubated overnight at 37C to allow the collagen solution to gel. On the following day, 10 μl of a 500 mM glyceraldehyde solution was added to all of the wells and the plate was incubated for 24 hours in a humidified incubator at 37° C. On the following day 40 μl of either test material (prepared in assay buffer), aminoguanidine (positive control), or assay buffer (negative control) was added to the plate. The plate was then read using a fluorometer (355 nm excitation and 405 nm emission) and then incubated again for 24 hours in a humidified incubator at 37C. On the following day the plate was read again. AGE formation was determined by subtracting the initial fluorescent measurement from the final fluorescent measurement for each sample.

c. DPPH Assay

The DPPH assay is based on the measurement of the scavenging effect of antioxidants on the stable radical 2,2-diphenyl-1-picrylhydrazyl (DPPH). The free radical DPPH has a strong absorbance at 490 nm, and this absorbance is reduced when DPPH reacts with antioxidant compounds and is converted to hydrazine. The DPPH assay is considered a valid and easy assay to evaluate scavenging activity of antioxidants, since the radical compound is stable and does not have to be generated as in other radical scavenging assays.

The Trolox standard and the test samples were prepared in methanol at 10× their final desired concentration. All concentrations reported in this study represent the final concentration of the sample in the assay. For the assay, 200 μl of 0.5 mM DPPH prepared in methanol was combined with 20 μl of test material or 20 μl of methanol (untreated sample) and allowed to incubate at room temperature for 30 minutes in the dark. After the 30-minute incubation, the samples were read at 490 nm.

d. Cyclobutane Pyrimidine Dimer Assay (Prevention of DNA Damage)

The testing system used for this assay was the MatTek EpiDerm, a skin model which consists of normal human-derived epidermal keratinocytes cultured to form a multilayered, highly differentiated model of the human epidermis. For this study, tissues were treated topically either overnight with the test materials prior to UVB exposure (pretreat group to assess prevention). Following treatment, DNA was extracted from the EpiDerm tissues and assayed for CPD content using an ELISA based method.

### Example 3

### Bioassay Results

a. Elastase and Collagenase Inhibition

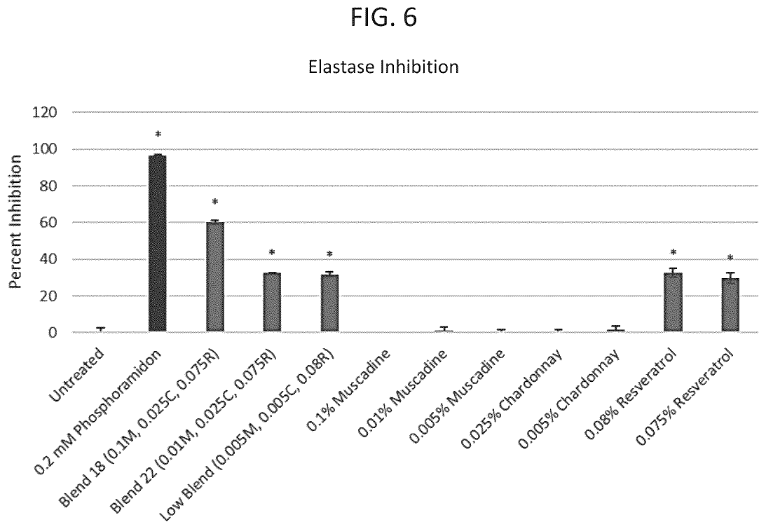

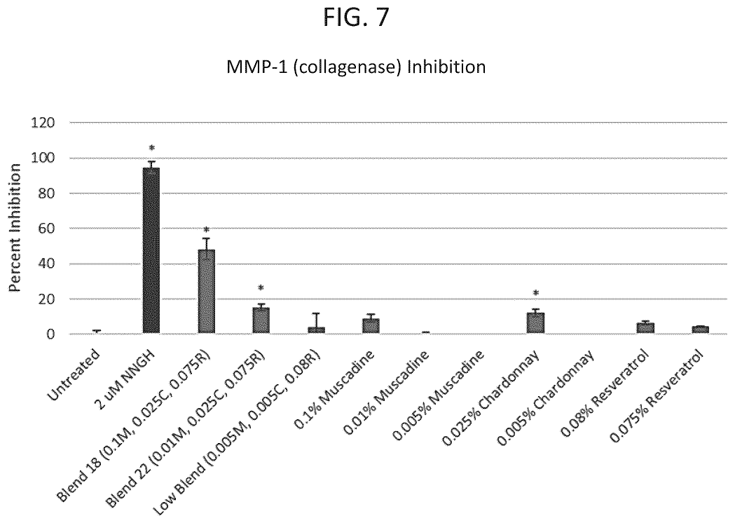

The purpose of this study was to determine if the test materials could inhibit elastase and collagenase. In this study both blend 18 (extract blend A) and 22 (extract blend B) were observed to inhibit both enzymes with blend 18 (extract blend A) being the most effective of the two (see, FIGS. 6 and 7; Tables 10 and 11). The third blend tested in this study, the Low Blend, was observed to inhibit elastase but not collagenase. With respect to the individual ingredients, resveratrol appears to be effective at inhibiting elastase activity on its own, while the Chardonnay extract appears to be effective at inhibiting collagenase on its own. There appears to be an increased effect in Blend 18 (extract blend A) in inhibiting elastase and collagenase that is greater than the net sum of the effects of the individual materials. This observation suggests that there is a synergistic effect between the materials in this blend such that they are more effective in combination with each other than they are alone. In the following tables, “M” denotes Muscadine Grape Extract (RM113), “C” denotes Chardonnay Grape Extract (YG093), “R” denotes Resveratrol (YR066), “2FD” denotes 2-fold dilution of an extract blend, “5FD” denotes 5-fold dilution of an extract blend, and “IOFD” denotes a 10-fold dilution of an extract blend. Additionally, test blend 18 is Extract Blend A and test blend 22 is Extract Blend B.

TABLE 10
Elastase Inhibition Data

| | Percent |
|---|---|
| Treatment | Inhibition |
| Untreated | 0 ± 3 |
| 0.2 mM Phosphoramidon | 97 ± 0* |
| Blend 18 (0.1M, 0.025C, 0.075R) | 60 ± 1* |
| Extract Blend A | |
| Blend 22 (0.01M, 0.025C, 0.075R) | 32 ± 0* |
| Extract Blend B | |
| Low Blend (0.005M, 0.005C, 0.08R) | 32 ± 1* |
| 0.1% Muscadine | −4 ± 5 |
| 0.01% Muscadine | 1 ± 2 |
| 0.005% Muscadine | 0 ± 2 |
| 0.025% Chardonnay | −1 ± 2 |
| 0.005% Chardonnay | 1 ± 2 |
| 0.08% Resveratrol | 33 ± 2* |
| 0.075% Resveratrol | 30 ± 3* |

*Significantly different from the Untreated group (p < 0.05).

TABLE 11
Collagenase Inhibition Data

| | Percent |
|---|---|
| Treatment | Inhibition |
| Untreated | 0 ± 2 |
| 2 uM NNGH | 94 ± 3* |
| Blend 18 (0.1M, 0.025C, 0.075R) | 48 ± 6* |
| Extract Blend A | |
| Blend 22 (0.01M, 0.025C, 0.075R) | 15 ± 2* |
| Extract Blend A | |
| Low Blend (0.005M, 0.005C, 0.08R) | 4 ± 8 |
| 0.1% Muscadine | 9 ± 2 |
| 0.01% Muscadine | −2 ± 2 |
| 0.005% Muscadine | −3 ± 2 |
| 0.025% Chardonnay | 12 ± 2* |
| 0.005% Chardonnay | −4 ± 2 |
| 0.08% Resveratrol | 7 ± 1 |
| 0.075% Resveratrol | 4 ± 0 |

*Significantly different from the Untreated group (p < 0.05).

b. Collagen AGE Prevention and Reduction

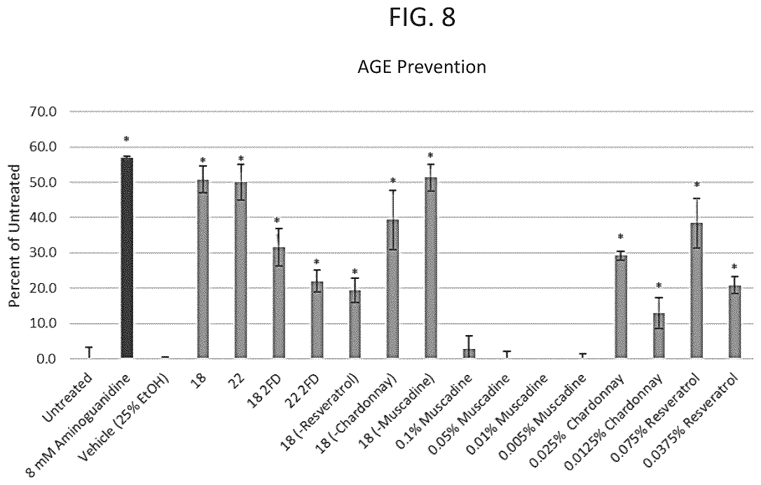

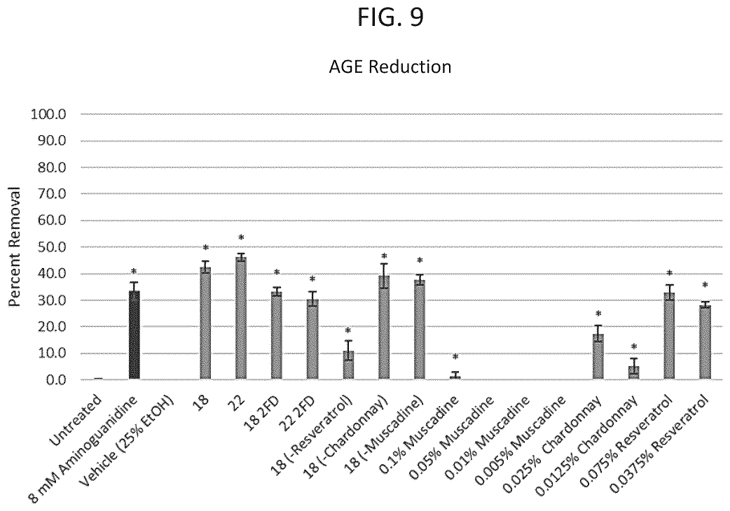

The purpose of this study was to assess the ability of the test materials to prevent AGE formation in collagen and to also determine if the materials could reduce preexisting AGEs in collagen. In this study, both blends 18 (Extract Blend A) and 22 (Extract Blend B) were effective at significantly preventing AGE formation and reducing existing AGEs (see, FIGS. 8 and 9; see also, Tables 12 and 13). This significant effect was observed with both the blends at full strength and as two-fold dilutions (2FD). In addition, when Blend 18 (Extract Blend A) was tested as combinations of just two of the three ingredients it was still observed to be effective, although with varying levels of effectiveness. With respect to both blends, when the ingredients were tested individually at the concentrations used in the blends, the chardonnay and resveratrol were still observed to be significantly effective. However, the muscadine ingredient was not observed to be effective at preventing AGE formation and only effective at reducing existing AGEs at the 0.1% concentration.

TABLE 12
Percent Collagen AGE Prevention

| | % | % | % | Percent |
|---|---|---|---|---|
| | Mus- | Char- | Res- | AGE |
| Treatment | cadine | donnay | veratrol | Prevention |
| Untreated | — | — | — | 0.0 ± 3.2 |
| 8 mM Aminoguanidine | — | — | — | 56.8 ± 0.5* |
| Vehicle (25% EtOH) | — | — | — | −1.1 ± 1.5 |
| Blend 18 | 0.1000 | 0.0250 | 0.0750 | 50.8 ± 3.8* |
| (0.1M, 0.025° C., 0.075R) | | | | |
| (Extract Blend A) | | | | |
| Blend 22 | 0.0100 | 0.0250 | 0.0750 | 50.0 ± 5.1* |
| (0.01M, 0.025C, 0.075R) | | | | |
| (Extract Blend B) | | | | |
| 18 2FD | 0.0500 | 0.0125 | 0.0375 | 31.6 ± 5.2* |
| 22 2FD | 0.0050 | 0.0125 | 0.0375 | 22.0 ± 3.0* |
| 18 (-Resveratrol) | 0.1000 | 0.0250 | 0.0000 | 19.3 ± 3.4* |
| 18 (-Chardonnay) | 0.1000 | 0.0000 | 0.0750 | 39.4 ± 8.4* |
| 18 (-Muscadine) | 0.0000 | 0.0250 | 0.0750 | 51.3 ± 3.8* |
| 0.1% Muscadine | 0.1000 | 0.0000 | 0.0000 | 2.7 ± 3.9 |
| 0.05% Muscadine | 0.0500 | 0.0000 | 0.0000 | −7.6 ± 9.8 |
| 0.01% Muscadine | 0.0100 | 0.0000 | 0.0000 | −2.6 ± 0.7 |
| 0.005% Muscadine | 0.0050 | 0.0000 | 0.0000 | −0.7 ± 2.1 |
| 0.025% Chardonnay | 0.0000 | 0.0250 | 0.0000 | 29.2 ± 1.2* |
| 0.0125% Chardonnay | 0.0000 | 0.0125 | 0.0000 | 13.0 ± 4.4* |
| 0.075% Resveratrol | 0.0000 | 0.0000 | 0.0750 | 38.4 ± 7.1* |
| 0.0375% Resveratrol | 0.0000 | 0.0000 | 0.0375 | 20.8 ± 2.4* |

*Significantly different from the Untreated group (p < 0.05)

TABLE 13
Percent Collagen AGE Reduction

| | % | % | % | Percent |
|---|---|---|---|---|
| | Mus- | Char- | Res- | AGE |
| Treatment | cadine | donnay | veratrol | Reduction |
| Untreated | — | — | — | −1.1 ± 1.3 |
| 8 mM Aminoguanidine | — | — | — | 33.4 ± 3.3* |
| Vehicle (25% EtOH) | — | — | — | −6.1 ± 0.8 |
| Blend 18 | 0.1000 | 0.0250 | 0.0750 | 42.5 ± 2.2* |
| Extract Blend A | | | | |
| Blend 22 | 0.0100 | 0.0250 | 0.0750 | 46.1 ± 1.4* |
| Extract Blend B | | | | |
| Blend 18 2FD | 0.0500 | 0.0125 | 0.0375 | 33.2 ± 1.5* |
| Extract Blend A | | | | |
| Blend 22 2FD | 0.0050 | 0.0125 | 0.0375 | 30.5 ± 2.8* |
| Extract Blend B | | | | |
| 18 (-Resveratrol) | 0.1000 | 0.0250 | 0.0000 | 11.0 ± 3.5* |
| 18 (-Chardonnay) | 0.1000 | 0.0000 | 0.0750 | 39.1 ± 4.5* |
| 18 (-Muscadine) | 0.0000 | 0.0250 | 0.0750 | 37.5 ± 1.9* |
| 0.1% Muscadine | 0.1000 | 0.0000 | 0.0000 | 1.3 ± 1.5* |
| 0.05% Muscadine | 0.0500 | 0.0000 | 0.0000 | −4.5 ± 2.2 |
| 0.01% Muscadine | 0.0100 | 0.0000 | 0.0000 | −5.0 ± 0.6 |
| 0.005% Muscadine | 0.0050 | 0.0000 | 0.0000 | −6.3 ± 1.5 |
| 0.025% Chardonnay | 0.0000 | 0.0250 | 0.0000 | 17.4 ± 3.0* |
| 0.0125% Chardonnay | 0.0000 | 0.0125 | 0.0000 | 5.2 ± 2.9* |
| 0.075% Resveratrol | 0.0000 | 0.0000 | 0.0750 | 32.8 ± 2.8* |
| 0.0375% Resveratrol | 0.0000 | 0.0000 | 0.0375 | 28.2 ± 1.2* |

*Significantly different from the Untreated group (p < 0.05).

c. DPPH Scavenging Assay

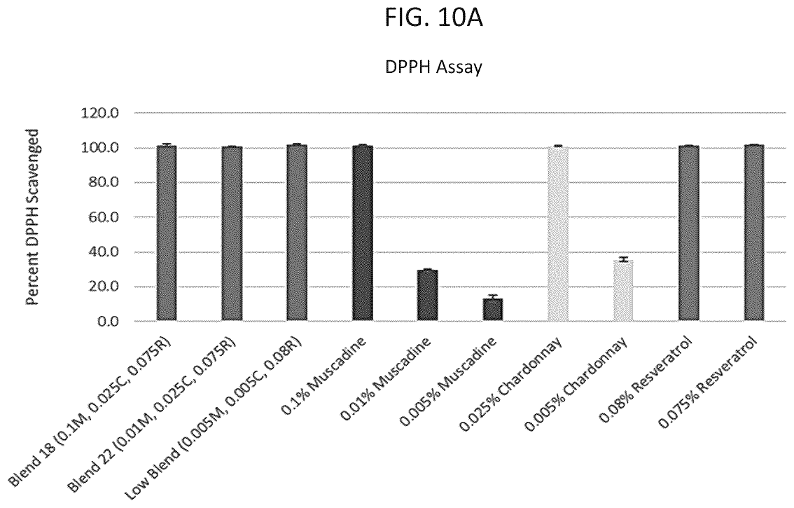

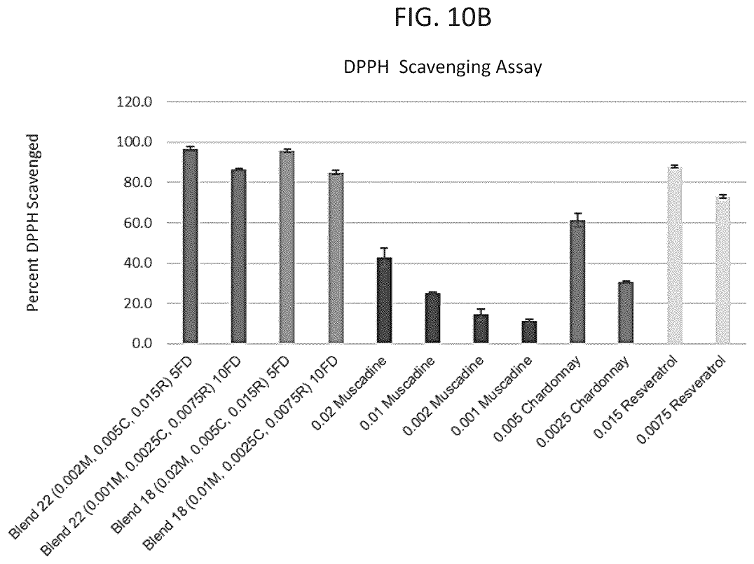

The purpose of this study was to screen blends of materials along with their individual components to determine the level of antioxidant capacity in each, with the goal of determining how the blends perform versus the performance of their individual ingredients. The first run of the study is presented in Table 14 (see also, FIG. 10A), which shows how well test materials scavenge the DPPH free radical. In this case, all three blends, the higher concentrations of the muscadine and chardonnay extract and both concentrations of the resveratrol completely scavenged all the DPPH free radicals (100% scavenged) which prevented any meaningful comparisons between the blends and their individual components. To address this, the three blends and their individual components were diluted either 5-fold or 10-fold and taken through the assay again (see, Table 15 and FIG. 10B). The antioxidant capacity of the blends was still high with the dilutions, but they did not reach the level of 100% DPPH scavenged. The dilutions also provided a better resolution of the antioxidant capacity for the muscadine and chardonnay extracts as well as the resveratrol.

TABLE 14
DPPH Assay Blends

| | Percent DPPH |
|---|---|
| Concentration (Percent) | Scavenged |
| Blend 18 (0.1M, 0.025° C., 0.075R) | 101.1 ± 1.0 |
| Extract Blend A | |
| Blend 22 (0.01M, 0.025C, 0.075R) | 100.7 ± 0.1 |
| Extract Blend B | |
| Low Blend (0.005M, 0.005C, 0.08R) | 101.9 ± 0.5 |
| 0.1% Muscadine | 101.3 ± 0.3 |
| 0.01% Muscadine | 29.5 ± 0.8 |
| 0.005% Muscadine | 13.2 ± 1.8 |
| 0.025% Chardonnay | 101.0 ± 0.2 |
| 0.005% Chardonnay | 35.6 ± 1.4 |
| 0.08% Resveratrol | 101.0 ± 0.3 |
| 0.075% Resveratrol | 101.5 ± 0.2 |

TABLE 15
DPPH Assay Diluted Blends

| | Percent DPPH |
|---|---|
| Concentration (Percent) | Scavenged |
| Blend 22—5FD (0.002M, 0.005C, | 96.7 ± 1.1 |
| 0.015R) Extract Blend B | |
| Blend 22—10 FD (0.001M, 0.0025C, | 86.5 ± 0.4 |
| 0.0075R) Extract Blend B | |
| Blend 18—5FD (0.02M, 0.005C, | 95.7 ± 1.0 |
| 0.015R) Extract Blend A | |
| Blend 18—10FD (0.01M, 0.0025C, | 84.9 ± 1.0 |
| 0.0075R) Extract Blend A | |
| 0.02 Muscadine | 42.6 ± 4.7 |
| 0.01 Muscadine | 25.2 ± 0.2 |
| 0.002 Muscadine | 14.4 ± 2.7 |
| 0.001 Muscadine | 11.0 ± 1.1 |
| 0.005 Chardonnay | 61.2 ± 3.5 |
| 0.0025 Chardonnay | 30.6 ± 0.4 |
| 0.015 Resveratrol | 87.9 ± 0.5 |
| 0.0075 Resveratrol | 73.1 ± 0.8 |

d. Cyclobutane Pyrimidine Dimer Assay (Prevention of DNA Damage)

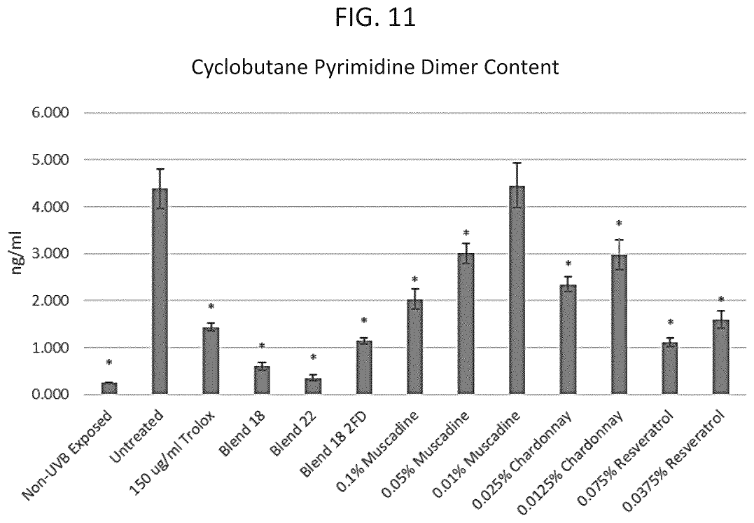

The purpose of this study was to determine if the test materials had an impact on UVB induced CPD formation in genomic DNA using MatTek EPIDERM® tissues as the model. In this study the two blends (Blend 18 and Blend 22) were the most effective treatment for preventing CPD formation (see, Table 16 and FIG. 11). In addition to the two blends, all of the individual components were observed to prevent CPD formation (with the exception of the 0.01% concentration of Muscadine) although the effectiveness of the individual materials was less than the effectiveness of the blends.

TABLE 16
Percent Prevention of Genomic DNA CPD Formation

| | % | % | % | | % |
|---|---|---|---|---|---|
| | Mus- | Char- | Res- | | Pre- |
| Treatment | cadine | donnay | veratrol | ng/ml | vention |
| Non-UVB | — | — | — | 0.261 ± 0.000* | — |
| Exposed | | | | | |
| Untreated | — | — | — | 4.386 ± 0.416 | 0 |
| 150 ug/ml | — | — | — | 1.439 ± 0.088* | 67 |
| Trolox | | | | | |
| Blend 18 | 0.1000 | 0.0250 | 0.0750 | 0.607 ± 0.088* | 86 |
| (Extract | | | | | |
| Blend A) | | | | | |
| Blend 22 | 0.0100 | 0.0250 | 0.0750 | 0.360 ± 0.065* | 92 |
| (Extract | | | | | |
| Blend B) | | | | | |
| Blend | 0.0500 | 0.0125 | 0.0375 | 1.143 ± 0.065* | 74 |
| 18 2FD | | | | | |
| 0.1% | 0.1000 | 0.0000 | 0.0000 | 2.031 ± 0.212* | 54 |
| Muscadine | | | | | |
| 0.05% | 0.0500 | 0.0000 | 0.0000 | 3.004 ± 0.213* | 32 |
| Muscadine | | | | | |
| 0.01% | 0.0100 | 0.0000 | 0.0000 | 4.450 ± 0.473 | −1 |
| Muscadine | | | | | |
| 0.025% | 0.0000 | 0.0250 | 0.0000 | 2.349 ± 0.162* | 46 |
| Chardonnay | | | | | |
| 0.0125% | 0.0000 | 0.0125 | 0.0000 | 2.983 ± 0.318* | 32 |
| Chardonnay | | | | | |
| 0.075% | 0.0000 | 0.0000 | 0.0750 | 1.115 ± 0.085* | 75 |
| Resveratrol | | | | | |
| 0.0375% | 0.0000 | 0.0000 | 0.0375 | 1.601 ± 0.180* | 63 |
| Resveratrol | | | | | |

*Denotes values which are significantly different from Untreated

e. Summary of Results

Extract Blends A and B showed statistically significant activity amongst a variety of lab-based in-vitro assays, including AGE, CPD, Collagenase, Elastase, and DPPH assays. The ratio of the blends used is directly linked to strong synergistic activity in the Collagenase and Elastase assay, specifically for Blend A, and possibly a synergistic activity on 2-fold dilutions for extract Blends A and B in the AGE reduction assay. In addition, variations of the extract blend ratios amongst these three components could generate desired synergistic activity in the Cyclobutane Pyrimidine Dimer, DPPH, and AGE prevention assays.

A variety of blend combinations were initially tested in the AGE prevention and reduction collagen assays to help determine efficacious topical composition for collagen prevention and reduction used in both the body serum and body lotion products, along with factoring in color implications for each product. The data clearly suggest the disclosed combination of ingredients together plays a pivotal role in a strong synergistic effect for the collagenase and elastase inhibition assays and modest synergistic effect in the AGE reduction assay. Furthermore, the overall effectiveness of the extract Blends A and B were greater than individual effectiveness of a single component of the blends, suggesting that blending these three components was complimentary and not inhibitory.

TABLE 17
Summary of in-vitro bioassay results

| Bioassay | Extract Blend A | Extract Blend B |
|---|---|---|
| Elastase | 60% Inhibition | 32% Inhibition |
| Collagenase | 48% Inhibition | 15% Inhibition |
| AGE—Reduction | 42.5% Reduction | 46.1% Reduction |
| AGE—Prevention | 50.8% Prevention | 50.0% Prevention |
| DPPH | 100% DPPH | 100% DPPH |
| | scavenged | scavenged |
| Cyclobutane | 86% Prevention | 92% Prevention |
| Pyrimidine Dimer | | |

TABLE 18
Summary of Synergistic Results in various in-vitro lab assays

| | | | AGE | |
|---|---|---|---|---|
| | Elastase | Collagenase | Collagen | |
| Sample | Inhibition | Inhibition | Reduction | Results |
| Extract | 60.0%—Actual | 48.0%—Actual | | 240% increase over |
| Blend A | 25.0%—Expected | 25.0% —Expected | | expected elastase |
| | | | | inhibition and 192% |
| | | | | increase over |
| | | | | expected collagenase |
| | | | | inhibition |
| Extract | | | 33.2%—Actual | 14.8% increase over |
| Blend A- | | | 28.9%—Expected* | expected AGE |
| 2FD | | | | reduction in collagen |
| Extract | | | 30.5%—Actual | 12.5% increase over |
| Blend B- | | | 27.1%—Expected* | expected AGE |
| 2FD | | | | reduction in collagen |

*Addition of separate components for each respective blend: RM113 + YG093 + YR066

### Example 4

### Exemplary Cosmetic Formulations

Listed below in Tables 19 and 20 are exemplary formulations for a Serum and Lotion. Extract Blend A is in the serum formula, whereas extract Blend B is in the lotion formula.

TABLE 19
Exemplary serum formula.

| Ingredient | Composition |
|---|---|
| Aqua | 85.993695 |
| Magnolia Officinalis (Houpu Magnolia) Bark Extract | 3.460000 |
| Glycerin | 3.010000 |
| Ammonium Acryloyldimethyltaurate/ VP Copolymer | 1.067000 |
| Dipalmitoyl Hydroxyproline | 1.000000 |
| Niacinamide | 1.000000 |
| Dicaprylyl Carbonate | 0.950000 |
| Linoleic Acid | 0.720000 |
| Fragrance | 0.600000 |
| Hydroxyacetophenone | 0.500000 |
| Polyglyceryl-3 Diisostearate | 0.500000 |
| Squalane | 0.500000 |
| Oleic Acid | 0.150000 |
| Vitis Rotundifolia (Muscadine Grape) Fruit Extract | 0.100000 |
| Palmitic Acid | 0.080000 |
| Resveratrol | 0.075000 |
| Carnosine | 0.060000 |
| Tocopherol (Vitamin E) | 0.051000 |
| Propanediol | 0.042000 |
| Stearic Acid | 0.040000 |
| T-Butyl Alcohol | 0.033000 |
| Vitis Vinifera (Chardonnay Grape) Seed Extract | 0.025000 |
| Ceramide NP | 0.010000 |
| Heptapeptide-7 | 0.008000 |
| Linolenic Acid | 0.005000 |
| Pentylene Glycol | 0.004000 |
| Behenic Acid | 0.002000 |
| Citric Acid | 0.002000 |
| Lecithin | 0.002000 |
| Potassium Sorbate | 0.002000 |
| Sodium Benzoate | 0.002000 |
| Xanthan Gum | 0.002000 |
| Arachidic Acid | 0.001000 |
| Gondoic Acid | 0.001000 |
| Sodium Acetylated Hyaluronate | 0.001000 |
| Sodium Hyaluronate | 0.000800 |
| Sodium Hyaluronate Crosspolymer | 0.000300 |
| Hydrolyzed Sodium Hyaluronate | 0.000150 |
| Ethylhexylglycerin | 0.000055 |
| Total: | 100.00 |

TABLE 20
Exemplary lotion formula.

| Ingredient | Composition |
|---|---|
| Aqua | 73.07810 |
| Glyceryl Stearate | 5.25000 |
| Dicaprylyl Carbonate | 3.80000 |
| Glycerin | 3.03503 |
| Butyrospermum Parkii (Shea) Butter | 2.50000 |
| Helianthus Annuus (Sunflower) Seed Oil | 2.00000 |
| Magnolia Officinalis (Houpu Magnolia) Bark Extract | 1.73000 |
| Lactobacillus | 1.60000 |
| Fragrance | 1.00000 |
| Cetearyl Alcohol | 0.75000 |
| Cetyl Palmitate | 0.75000 |
| Cocoglycerides | 0.75000 |
| Linoleic Acid | 0.72000 |
| Polyacrylate Crosspolymer-6 | 0.57000 |
| Prunus Domestica (Plum) Seed Extract | 0.50000 |
| Cocos Nucifera (Coconut) Fruit Extract | 0.40000 |
| Benzoic Acid | 0.29000 |
| Hydroxyacetophenone | 0.25000 |
| Tocopherol (Vitamin E) | 0.201000 |
| Oleic Acid | 0.15000 |
| Avena Sativa (Oat) Kernel Oil | 0.10000 |
| Niacinamide | 0.10000 |
| Tocopheryl Acetate (Vitamin E) | 0.10000 |
| Allantoin | 0.09990 |
| Palmitic Acid | 0.08000 |
| Resveratrol | 0.07500 |
| Stearic Acid | 0.04000 |
| Vitis Vinifera (Chardonnay Grape) Seed Extract | 0.02500 |
| Propanediol | 0.02000 |
| T-Butyl Alcohol | 0.01200 |
| Vitis Rotundifolia (Muscadine Grape) Fruit Extract | 0.01000 |
| Linolenic Acid | 0.00500 |
| Behenic Acid | 0.00200 |
| Arachidic Acid | 0.00100 |
| Gondoic Acid | 0.00100 |
| Gluconolactone | 0.000800 |
| Hamamelis Virginiana (Witch Hazel) Extract | 0.000600 |
| Melissa Officinalis (Lemon Balm) Leaf Extract | 0.000600 |
| Rosmarinus Officinalis (Rosemary) Leaf Extract | 0.000600 |
| Salvia Officinalis (Sage) Leaf Extract | 0.000600 |
| Thymus Vulgaris (Thyme) Flower/Leaf Extract | 0.000600 |
| Phenethyl Alcohol | 0.000305 |
| Potassium Sorbate | 0.000300 |
| Sodium Benzoate | 0.000300 |
| Caprylyl Glycol | 0.000250 |
| Calcium Gluconate | 0.000012 |
| Total: | 100.00 |

### Example 5

### Clinical Trial

In one clinical trial, at least 20 participants (e.g., 30 participants) are instructed to apply either a disclosed serum (e.g., the serum of Example 4, Table 19) or a disclosed body lotion (e.g., the lotion of Example 4, Table 20) once a day for 30 days. In another clinical trial, at least 20 participants are instructed to apply both the serum and the body lotion (with the serum applied first and lotion applied second) once a day for 30 days. Participants are asked to complete self-perception questionnaires (SPQs) at 2 weeks and at 30 days. Participants are asked to provide reports on skin firmness, elasticity, and hydration prior to application (baseline), at 2 weeks, and at 30 days.

It will be apparent that the precise details of the methods or compositions described herein may be varied or modified without departing from the spirit of the disclosure. We claim all such modifications and variations that fall within the scope and spirit of the claims below.

We claim:

1. A topical composition, comprising:
(a) a decolorized muscadine grape pomace extract at about 0.025% to about 0.4% by weight of the topical composition (w/w), a water extract of Chardonnay grape seed at about 0.006% to about 0.1% by weight of the topical composition (w/w), and resveratrol at about 0.018% to about 0.3% by weight of the topical composition (w/w); or
(b) the decolorized muscadine grape pomace extract at about 0.0025% to about 0.04% by weight of the topical composition (w/w), the water extract of Chardonnay grape seed at about 0.006% to about 0.1% by weight of the topical composition (w/w), and resveratrol at about 0.018% to about 0.3% by weight of the topical composition (w/w),
wherein for both (a) and (b), the decolorized muscadine grape pomace extract comprises by percentage dry weight of the extract about 9% to about 10% polyphenols and less than 4% monosaccharides, and wherein condensed tannins are less than 10% of the polyphenols of the decolorized muscadine grape pomace extract, and
wherein for both (a) and (b), the water extract of Chardonnay grape seed comprises by percentage dry weight of the water extract 38-50% polyphenols, 9-12% fiber, 1-2% protein, <1% lipids, and 25-30% sugars.

2. The topical composition of claim 1, comprising (a) the decolorized muscadine grape pomace extract at about 0.025% to about 0.4% w/w, the water extract of Chardonnay grape seed at about 0.006% to about 0.1% w/w, and resveratrol at about 0.018% to about 0.3% w/w.

3. The topical composition of claim 2, comprising:
(i) the decolorized muscadine grape pomace extract at about 0.05% to about 0.2% w/w, the water extract of Chardonnay grape seed at about 0.0125% to about 0.05% w/w, and resveratrol at about 0.0375% to about 0.15% w/w;
(ii) the decolorized muscadine grape extract at about 0.10% w/w, the water extract of Chardonnay grape seed at about 0.025% w/w, and resveratrol at about 0.075% w/w; or
(iii) the decolorized muscadine grape extract at about 0.05% w/w, the water extract of Chardonnay grape seed at about 0.0125% w/w, and resveratrol at about 0.0375% w/w.

4. The topical composition of claim 1, comprising (b) the decolorized muscadine grape pomace extract at about 0.0025% to about 0.04% w/w, the water extract of Chardonnay grape seed at about 0.006% to about 0.1% w/w, and resveratrol at about 0.018% to about 0.3% w/w.

5. The topical composition of claim 4, comprising:
(i) the decolorized muscadine grape pomace extract at about 0.005% to about 0.02% w/w, the water extract of Chardonnay grape seed at about 0.0125% to about 0.05% w/w, and resveratrol at about 0.0375% to about 0.15% w/w;
(ii) the decolorized muscadine grape pomace extract at about 0.01% w/w, the water extract of Chardonnay grape seed at about 0.025% w/w, and resveratrol at about 0.075% w/w; or
(iii) the decolorized muscadine grape pomace extract at about 0.005% w/w, the water extract of Chardonnay grape seed at about 0.0125% w/w, and resveratrol at about 0.0375% w/w.

6. A topical composition, comprising a blend of decolorized muscadine grape pomace extract, water extract of Chardonnay grape seed, and resveratrol, in a ratio of 1-10:1-5:2.5-7.5, respectively,
wherein the decolorized muscadine grape pomace extract comprises by percentage dry weight of the extract about 9% to about 10% polyphenols and less than about 4% monosaccharides, and wherein condensed tannins are less than about 10% of the polyphenols of the decolorized muscadine grape pomace extract, and
wherein the water extract of Chardonnay grape seed comprises by percentage dry weight of the water extract 38-50% polyphenols, 9-12% fiber, 1-2% protein, <1% lipids, and 25-30% sugars.

7. The topical composition of claim 6, comprising the blend in a ratio of:
(i) 1-10:2.5:7.5, respectively;
(ii) 10:2.5:7.5, respectively; or
(iii) 1:2.5:7.5, respectively.

8. The topical composition of claim 1, wherein the topical composition is a cosmetic, optionally wherein the cosmetic is a serum or lotion.

9. The topical composition of claim 3, wherein the topical composition is a serum.

10. The topical composition of claim 5, wherein the topical composition is a lotion.

11. The topical composition of claim 1, further comprising one or more of: vitamins, additional botanical extracts, fats or oils, surfactants, emollients, emulsifiers, humectants, moisturizers, prebiotics, probiotics, and enzymes.

12. The topical composition of claim 1, wherein the topical composition inhibits elastase or matrix metalloproteinase-1 (MMP-1), decreases advanced glycation end product (AGE) formation or decreases AGE content of collagen, inhibits collagen breakdown, or improves skin appearance, as compared to a control.

13. A method of inhibiting elastase or matrix metalloproteinase-1 (MMP-1) in skin, decreasing advanced glycation end product (AGE) formation or decreasing AGE content of collagen in skin, decreasing collagen breakdown in skin, or improving appearance of skin, in a subject, comprising:
applying to the skin of the subject an effective amount of the topical composition of claim 1, thereby inhibiting elastase or MMP-1, decreasing AGE formation or decreasing AGE content of collagen, decreasing collagen breakdown, or improving appearance of skin, in the subject.

14. A method of inhibiting elastase or matrix metalloproteinase-1 (MMP-1) in skin of a subject by greater than 25%, comprising: applying to the skin of the subject an effective amount of the topical composition claim 3, thereby inhibiting elastase or MMP-1 in the skin of the subject.

15. A method of decreasing advanced glycation end product (AGE) formation or decreasing AGE content of collagen in skin of a subject by at least 30%, comprising: applying to the skin of the subject an effective amount of the topical composition of claim 3, thereby decreasing AGE formation or decreasing AGE content of collagen in the skin of the subject.

16. A method of decreasing advanced glycation end product (AGE) formation or decreasing AGE content of collagen in skin of a subject by at least 30%, comprising: applying to the skin of the subject an effective amount of the topical composition of claim 5, thereby decreasing AGE formation or decreasing AGE content of collagen in the skin of the subject.

17. The method of claim 13, wherein improving the appearance of the skin comprises at least one of: increasing skin firmness, increasing skin elasticity, or increasing skin hydration, as compared to a control.

18. The method of claim 13, wherein the topical composition is applied to the skin:
a) at least once a day for at least 30 days;
b) at least twice a day for at least 30 days;
c) at least once a day for at least 60 days; or
d) at least once a day for at least 90 days.

19. A method of preparing a topical composition, comprising:
providing a topical composition base,
mixing with the topical composition base:
I) a decolorized muscadine grape pomace extract at either:
a) about 0.025% to about 0.4% by weight of the topical composition (w/w), or
b) about 0.0025% to about 0.04% by weight of the topical composition (w/w);
II) a water extract of Chardonnay grape seed at about 0.006% to about 0.1% by weight of the topical composition (w/w); and
III) resveratrol at about 0.018% to about 0.3% by weight of the topical composition (w/w);
wherein the decolorized muscadine grape pomace extract comprises by percentage dry weight of the extract about 9% to about 10% polyphenols and less than 4% monosaccharides, and wherein condensed tannins are less than 10% of total polyphenols of the decolorized muscadine grape pomace extract, and
wherein the water extract of Chardonnay grape seed comprises by percentage dry weight of the water extract 38-50% polyphenols, 9-12% fiber, 1-2% protein, <1% lipids, and 25-30% sugars.

20. The method of claim 19, wherein the decolorized muscadine grape pomace extract is:
(i) at about 0.025% to about 0.4% w/w; or
(ii) at about 0.0025% to about 0.04% w/w.
